(19) United States
(12) Patent Application Publication
(10) Pub. No.: US 2026/0240856 A1
(43) Pub. Date: Aug. 20, 2026

(54) COMBINATION CANCER THERAPIES USING HETEROAROTINOIDS AND MYC INHIBITORS

(71) Applicant: The Board of Regents of the University of Oklahoma, Norman, OK (US)

(72) Inventors: Rajani RAI, Edmond, OK (US); Vishal CHANDRA, Edmond, OK (US); Doris BENBROOK, Oklahoma City, OK (US)

(21) Appl. No.: 19/537,855

(22) Filed: Feb. 12, 2026

Related U.S. Application Data
(60) Provisional application No. 63/758,637, filed on Feb. 14, 2025.

(51) Int. Cl. A61K 31/519; A61K 9/00; A61K 45/06

(52) U.S. Cl. CPC A61K 31/519; A61K 9/0019; A61K 45/06; A61K 2300/00

(57) ABSTRACT

Drug combinations of at least one heteroarotinoid (e.g., SHetA2, SHetA3, SHetA4, SHetC2, SHetD3, SHetD4, SHetD5, SHet50, SHet65, SHet100, OHet72, NHetl7, NHet86, or NHet90), and at least one MYC inhibitor such as a c-MYC inhibitor which are synergistically-effective as anti-cancer treatments, and kits and methods of use of such drug combinations.

### CROSS-REFERENCE TO RELATED APPLICATIONS

The present application claims the benefit under 35 USC § 119(e) of U.S. Provisional Patent Application No. 63/758,637, filed Feb. 14, 2025. The entire disclosure of the above-referenced patent application(s) are hereby expressly incorporated herein by reference.

### GOVERNMENT SUPPORT

Not Applicable.

### BACKGROUND

Cancer constitutes a significant health burden accounting for one in six deaths worldwide. According to Global Cancer Statistics, an estimated 20 million cancer cases were newly diagnosed in 2022 and 9.7 million people died from the disease worldwide. By 2050, the number of cancer cases is predicted to reach 35 million. Current state of treatment for cancer include surgical resection of the tumors followed by radiotherapy and/or chemotherapy. Surgery is mostly effective when patients are diagnosed at an early stage of disease progression while radiation therapy damages healthy cells, organs, and tissues. Although chemotherapy has reduced morbidity and mortality, it affects healthy cells, especially rapidly dividing and growing cells thereby causing severe side effects. Toxicity due to chemotherapy and radiation therapy burdens cancer patients with onerous physical and psychological challenges and negatively affects the quality of life. Furthermore, drug resistance, due to reduced drug uptake and increased drug efflux is another clinical challenge causing majority of patients uncurable.

Recent addition of bevacizumab and immune therapy have shown some improvement in overall survival of cancer patients however there are limited to only a subset of patients and expensive due to competitive in their production involving complex series of tissue culture and animal work. Therefore, there is an unmet clinical need to develop new molecularly targeted therapy which is less toxic and can improve dismal outcomes in cancer patients such as ovarian and cervical cancer patients.

### BRIEF DESCRIPTION OF THE DRAWINGS

Several embodiments of the present disclosure are hereby illustrated in the appended drawings. It is to be noted however, that the appended drawings only illustrate several typical embodiments and are therefore not intended to be considered limiting of the scope of the inventive concepts disclosed herein. The patent or application file contains at least one drawing executed in color. Copies of this patent or patent application publication with color drawing(s) will be provided by the Office upon request and payment of the necessary fee.

FIG. 1 is a scheme showing the mechanism of action of SHetA2.

FIG. 2A shows that SHetA2 reduces viability in several cell lines of cervical cancer (MTT assay, 72 hr).

FIG. 2B shows that SHetA2 reduces tumor growth in CaSki and SiHa cervical cancer xenografts (2-way ANOVA).

FIG. 2C shows that SHetA2 damages mitochondrial structure in SiHa cervical cancer cells and CaSki xenograft tumor cell (TEM imaging, M: mitochondria, N: nucleus).

FIG. 2D shows that SHetA2 inhibits mitochondrial networking in cervical cancer cells (confocal microscopy analyzed by Image J software).

FIG. 2E shows that SHetA2 inhibits OCR in CaSki and SiHa cells.

FIG. 2F shows that SHetA2 upregulates ECAR in CaSki and SiHa cells.

FIG. 2G shows that SHetA2 shifts energetic maps toward glycolysis.

FIG. 3A demonstrates that SHetA2 induces glycolysis. LC-MS/MS analysis of SiHa cells treated with SHetA2 vs. control demonstrated significant increase in glycolytic intermediate metabolites (shown in boxes).

FIG. 3B shows results of pathway enrichment analysis of metabolomics data. The top altered pathways are indicated.

FIG. 4A shows that treatment of cervical and ovarian cancer cells with SHetA2 increases c-Myc expression after 48 h (WT: wild type, CPR: cisplatin-resistant OVCAR8 cells, ASC: Ascites spheroids).

FIG. 4B shows that treatment of mice having cervical xenograft tumors with SHetA2 increases c-Myc expression in the xenograft tumors.

FIG. 4C shows that a combination treatment of cancer cells and ovarian cancer ascites (ASCs) with SHetA2 and Myci975 resulted in a synergistic effect as indicated by combination index values (CI<1 is synergism; CI>1 is antagonism by Chou-Talalay method).

FIG. 5A shows that the treatment combination of SHetA2 and c-Myc inhibitor increased cleaved PARP expression in cervical cancer cells as compared to the single drug treatments (24 h).

FIG. 5B shows that the treatment combination of SHetA2 and c-Myc inhibitor increased caspase 3/7 activity in cervical cancer cells as compared to the single drug treatments (24 h).

FIG. 5C shows that the treatment combination of SHetA2 and c-Myc inhibitor inhibited clonogenic activity in cervical cancer cells. as compared to the single drug treatments (24 h).

FIG. 5D shows that the treatment combination of SHetA2 and c-Myc inhibitor inhibited migration ability in a wound gap assay in cervical cancer cells as compared to the single drug treatments (24 h).

FIG. 6A shows that a combination of SHetA2 and Myci975 synergistically inhibited proliferation and induces apoptosis in OvCa cells and ascites spheroids (ASC) as indicated by decreased expression of Cyclin D1 and increased expression of cleaved (cl.) PARP and YH2AX (WT: wild type, CPR: cisplatin-resistant-OVCAR8 cells, ASC: Ascites spheroids).

FIG. 6B shows that a combination of SHetA2 and Myci975 synergistically inhibited proliferation and induces apoptosis in OvCa cells and ASC as indicated by increased caspase activity (WT: wild type, CPR: cisplatin-resistant-OVCAR8 cells, ASC: Ascites spheroids).

FIG. 6C shows the effect of SHetA2 and Myci975 combination treatment on OvCa spheroid viability (WT: wild type, CPR: cisplatin-resistant-OVCAR8 cells, ASC: Ascites spheroids).

FIG. 7 shows the effects of a combination treatment of SHetA2 and MYCi975 on body weight of mice with SiHa cervical cancer xenograft tumors. Mice were treated with vehicle, SHetA2, MYCi975, or the combination of SHetA2 and MYCi975.

FIG. 8 shows the effects of a combination treatment of SHetA2 and MYCi975 on Tumor volume measurements over time in mice with SiHa cervical cancer xenograft tumors. Mice were treated with vehicle, SHetA2, MYCi975, or a combination of SHetA2 and MYCi975.

FIG. 9 shows the effects of a combination treatment of SHetA2 and MYCi975 on endpoint tumor weights in mice with SiHa cervical cancer xenograft tumors. Mice were treated with vehicle, SHetA2, MYCi975, or a combination of SHetA2 and MYCi975.

FIG. 10 shows the effects of a combination treatment of SHetA2 and MYCi975 on body weight of mice with OVCAR8-CPR ovarian cancer xenograft tumors. Mice were treated with vehicle, SHetA2, MYCi975, or a combination of SHetA2 and MYCi975.

FIG. 11 shows the effects of a combination treatment of SHetA2 and MYCi975 on endpoint tumor weights in mice with OVCAR8-CPR ovarian cancer xenograft tumors. Mice were treated with vehicle, SHetA2, MYCi975, or a combination of SHetA2 and MYCi975.

### ABBREVIATIONS

ANOVA: Analysis of Variance,
ASC: Ascites spheroids,
CI: Combination index,
CPR: cisplatin-resistant,
DRI: Dose-reduction index,
ECAR: Extracellular acidification rate,
FDA: Food and Drug Administration,
HET: heteroarotinoid,
HGSOC: High-grade serous ovarian cancer,
hFTSECs: Human fallopian tube Secretory epithelial cells,
IC: Inhibitory concentration,
IC50: Half maximal inhibitory concentration,
LC-MS: Liquid chromatography-Mass spectrometry;
M: mitochondria,
N: nucleus,
NSCLC: non-small-cell lung cancer,
OCR: Oxygen consumption rate,
PARP: Poly ADP ribose polymerase,
PDAC: pancreatic ductal adenocarcinoma,
SHetA2: Sulfur heteroarotinoid A2, ([(4-nitrophenyl)amino][2,2,4,4-tetramethylthiochroman-6-yl]amino]methane-thione),
OS: Overall Survival,
ROS: Reactive oxygen species,
SCLC: small-cell lung cancer,
TEM: Transmission electron microscopy,
TNBC: Triple-negative breast cancer,
WT: Wild type,
ZIP: Zero Interaction Potency.

### DETAILED DESCRIPTION

The present disclosure demonstrates that combinations of SHetA2 and MYC inhibitors decrease cancer burden by altering cancer cell metabolism without causing undue side effects to the patients. This novel drug combination therapy will improve the overall outcomes of cancer patients by providing new low-toxicity to no-toxicity therapeutic options, which are also pertinent for advanced and recurrent cancers because they target cancer-specific signaling pathways involved in drug resistance. MYC inhibitors may inhibit MYC protein or MYC protein expression. The MYC inhibitors may be c-MYC inhibitors. Examples of cancer types that can be treated using the combination therapies disclosed herein are cancers having MYC activation and/or overexpression and include, but are not limited to, leukemia, lymphoma, multiple myeloma, pancreatic ductal adenocarcinoma (PDAC), brain cancer, non-small-cell lung cancer (NSCLC), small-cell lung cancer (SCLC), liver cancer, prostate cancer, ovarian cancer, cervical cancer, TNBC, lung squamous carcinoma, lung adenocarcinoma, hepatocellular carcinoma, cutaneous melanoma, colon adenocarcinoma, bladder adenocarcinoma, prostate adenocarcinoma, breast adenocarcinoma, gastric adenocarcinoma, pancreatic adenocarcinoma, esophageal adenocarcinoma, ovarian adenocarcinoma, endometrial adenocarcinoma, uterine carcinosarcoma, acute myeloid leukemia, and diffuse large B cell lymphoma. Other cancers that can be treated are listed below.

Extensive preclinical studies of SHetA2 (Sulfur heteroarotinoid A2, a.k.a., NSC726189) have shown that this drug is not toxic. As of this date, SHetA2 is in Phase I testing (NCT04928508) and to date this trial has completed the third dose level and there is no evidence of toxicity (Korean Society of Medical Oncology 2024 meeting). Extensive preclinical studies showed that SHetA2 does not cause toxicity, cardiac effects, teratogenicity, or mutagenicity. The No Observed Adverse Effect Level of 28-day oral SHetA2 in dogs was 1,500 mg/kg/day representing a very large therapeutic window compared to the SHetA2 dose range (10-60 mg/kg/day) shown to reduce in vivo tumor growth. Targeting MYC with the genetically expressed modulator Omomyc (OMO-103) or small-molecule inhibitors have been shown to have mild, fully reversible, or no side effects to normal tissues. The first-in-human phase 1 trial of OMO-103, a mini-protein targeting the MYC oncogene, demonstrated promising antitumor activity and safety and is now moved to a phase 1b combination study in metastatic pancreatic cancer patients. Results of the present work demonstrated that MYC inhibitor complimented SHetA2 efficacy. The reversibility of MYC inhibitor side effects combined with the lack of SHetA2 toxicity in extensive preclinical studies indicates this combination treatment will be effective in treating cancer patients without causing severe side effects associated with currently used chemotherapy regimens.

Before further detailed description of various embodiments of the compositions and methods of use thereof of the present disclosure, it is to be understood that the present disclosure is not limited in application to the details of methods and compositions as set forth in the following description. The description provided herein is intended for purposes of illustration only and is not intended to be construed in a limiting sense. The present disclosure is capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that various embodiments of the present disclosure may be practiced without these specific details. In other instances, features which are well known to persons of ordinary skill in the art have not been described in detail to avoid unnecessary complication of the description. It is intended that all alternatives, substitutions, modifications and equivalents apparent to those having ordinary skill in the art are included within the scope of the present disclosure as defined herein. Thus the examples described below, which include particular embodiments, will serve to illustrate the practice of the present disclosure, it being understood that the particulars shown are by way of example and for purposes of illustrative discussion of particular embodiments only and are presented in the cause of providing what is believed to be a useful and readily understood description of procedures as well as of the principles and conceptual aspects of the inventive concepts. Thus, while the compositions and methods of the present disclosure have been described in terms of particular embodiments, it will be apparent to those of skill in the art that variations may be applied to the compositions and/or methods and in the steps or in the sequence of steps of the method described herein without departing from the concept, spirit, and scope of the inventive concepts disclosed herein.

Each patent, published patent application, and non-patent publication referenced in any portion of this application, including but not limited to U.S. Ser. No. 18/145,334, filed Dec. 22, 2022, U.S. Ser. No. 17/276,032, filed Mar. 12, 2021, International Application No. PCT/US2019/050775, filed Sep. 12, 2019, U.S. Ser. No. 62/730,345, filed Sep. 12, 2018, and U.S. Ser. No. 63/758,637, filed Feb. 14, 2025, is expressly incorporated herein by reference in its entirety to the same extent as if the individual patent, or published patent application, or non-patent publication was specifically and individually indicated to be incorporated by reference.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

As utilized in accordance with the methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example.

As noted above, any numerical range listed or described herein is intended to include, implicitly or explicitly, any number or sub-range within the range, particularly all integers, including the end points, and is to be considered as having been so stated. For example, “a range from 1.0 to 10.0” is to be read as indicating each possible number, including integers and fractions, along the continuum between and including 1.0 and 10.0, that is, having a minimum value equal to or greater than 1.0 and a maximum value equal to or less than 10.0, such as, for example, 3.25 to 8.65. Any maximum numerical limitation recited herein is intended to include all lower numerical limitations subsumed therein, and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein. Thus, even if a particular data point within the range is not explicitly identified or specifically referred to, it is to be understood that any data points within the range are to be considered to have been specified, and that the inventor(s) possessed knowledge of the entire range and the points within the range.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error for the composition, the method used to administer the composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, observer error, and combinations thereof, for example. The term “about” or “approximately”, where used herein when referring to a measurable value such as an amount, a temporal duration, and the like, is meant to encompass, for example, variations of ±20%, or +10%, or ±5%, or +1%, or +0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 80% of the time, at least 90% of the time, at least 91% of the time, at least 92% of the time, at least 93% of the time, at least 94% of the time, at least 95% of the time, at least 96% of the time, at least 97% of the time, at least 98% of the time, or at least 99% of the time.

As used herein any reference to “one embodiment” or “an embodiment” means that a particular element, feature, composition, structure, or characteristic described in connection with the embodiment is included in at least one embodiment. The appearances of the phrase “in one embodiment” in various places in the specification are not necessarily all referring to the same embodiment.

The term “mutant” or “variant” is intended to refer to a protein, peptide, nucleic acid or organism which has at least one amino acid or nucleotide which is different from the wild type version of the protein, peptide, nucleic acid, or organism and includes, but is not limited to, point substitutions, multiple contiguous or non-contiguous substitutions, chimeras, or fusion proteins, and the nucleic acids which encode them. Examples of conservative amino acid substitutions include, but are not limited to, substitutions made within the same group such as within the group of basic amino acids (such as arginine, lysine, histidine), acidic amino acids (such as glutamic acid and aspartic acid), polar amino acids (such as glutamine and asparagine), hydrophobic amino acids (such as leucine, isoleucine, and valine), aromatic amino acids (such as phenylalanine, tryptophan, tyrosine) and small amino acids (such as glycine, alanine, serine, threonine, methionine). Other examples of possible substitutions are described below.

The term “pharmaceutically acceptable” refers to compounds and compositions which are suitable for administration to humans and/or animals without undue adverse side effects such as toxicity, irritation and/or allergic response commensurate with a reasonable benefit/risk ratio.

As used herein, “pure,” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species (e.g., the active agent) is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

The terms “subject” and “patient” are used interchangeably herein and will be understood to refer to a warm blooded animal, particularly a mammal. Non-limiting examples of animals within the scope and meaning of this term include dogs, cats, rabbits, rats, mice, guinea pigs, chinchillas, hamsters, ferrets, horses, pigs, goats, cattle, sheep, zoo animals, camels, llamas, non-human primates, including Old and New World monkeys and non-human primates (e.g., cynomolgus macaques, chimpanzees, rhesus monkeys, orangutans, and baboons), and humans.

The term “active agent” where used herein is intended to refer to a substance which possesses a biological activity relevant to the present disclosure, and particularly refers to therapeutic and diagnostic substances which may be used in methods described in the present disclosure. By “biologically active” is meant the ability to modify the physiological system of a cell, tissue, or organism without reference to how the active agent has its physiological effects. Where used herein, unless otherwise noted, the term “active agent” includes pharmaceutically-acceptable salts, hydrates, solvates, and amorphous solids thereof.

Where used herein the terms heteroarotinoid and MYC inhibitor, and specific examples thereof, are intended to include pharmaceutically-acceptable salts, hydrates, solvates, and amorphous solids thereof.

Therapeutic drug combinations of the present disclosure include, but are not limited to, at least one heteroarotinoid, particularly flexible heteroarotinoids such as those further described below, and at least one MYC inhibitor.

Examples of compounds which may be used herein as MYC inhibitors in combination with the heteroarotinoids of the present disclosure (for example SHetA2), include but are not limited to: OMO-103 (Omomyc), MYCi975, MYCi361, 10058-F4, 10074-G5, MYCMI-6, BTYNB, EN4, APTO-253, CX5461, ML327, Mycro 3, KJ Pyr 9, SaJM589, KSI-3716, KI-MS2-008, Triptolide, WBC100 (14-dextrorotary-valyl-triptolide), MLN8237 (alisertib), CD532, BI-6727 (a.k.a. Volasertib), JQ1, OTX015/MK-8628, GSK2820151, ZEN-3694, CC-90010, ABBV-744, ABBV-075 (mivebresib), BI-894999, CPI-0610 (Pelabresib), GSK525762/I-BET762, INCB057643, dBET6, Alobresib, Inobrodib, BI-2536, LB-100, SMAP(DT-061), FTY720, OP449, perphenazine, KPT-6566, ATRA, BJP-06-005-3, Sulfopin, AZD1208, TP-3654, PIM447, SGI-1776, LGH447, SEL24/MEN1703, Everolimus, Paxalisib, ATG-008, Gedatolisib, Samotolisib, Sapanisertib, Apitolisib, Zotatifin, TAK-981, MRT-2359, and combinations thereof. In certain embodiments, each of these compounds may be used in combination with SHetA2. These MYC inhibitor compounds are discussed in further detail below.

OMO-103 is the first MYC inhibitor to enter clinical trials. The first-in-human phase I trial of OMO-103 demonstrates its safety and promising anti-tumor activity. OMO-103 is a mini-protein of 91 amino acids that inhibits MYC-MAX binding that are not able to bind DNA, thereby shutting down the transcription of MYC/MAX target genes.

MYCi975 (a.k.a. NUCC-0200975) is a potent, selective and orally active inhibitor of MYC that disrupts MYC/MAX interaction, promotes MYC T58 phosphorylation and MYC degradation, and impairs MYC driven gene expression. MYCi975 exhibits potent anti-tumor activities.

MYCi361 is an MYC inhibitor that engages MYC inside cells, disrupts MYC/MAX dimers, and impairs MYC-driven gene expression. MYCi361 binds to MYC. MYCi361 suppresses in vivo tumor growth, increases tumor immune cell infiltration, upregulates PD-L1 on tumors, and sensitizes tumors to anti-PD1 immunotherapy.

10058-F4 is a c-Myc inhibitor that specifically inhibits the c-Myc-Max interaction and prevents transactivation of c-Myc target gene expression. 10058-F4 promotes a caspase-3-dependent apoptosis and modulates autophagy.

10074-G5 is a c-Myc inhibitor that binds to and distorts the bHLH-ZIP domain of c-Myc, thereby inhibiting c-Myc/Max heterodimer formation and inhibiting its transcriptional activity.

MYCMI-6 (a.k.a. NSC354961) is a potent and selective endogenous inhibitor of MYC:MAX protein interactions. MYCMI-6 blocks MYC-driven transcription and binds to the MYC bHLHZip domain. MYCMI-6 inhibits tumor cell growth in a MYC-dependent manner and induces apoptosis.

BTYNB (a.k.a. BTYNB IMP1 Inhibitor, MDK6620) is a potent and selective inhibitor of IMP1 binding to c-Myc mRNA. BTYNB destabilizes c-Myc mRNA, resulting in downregulation of c-Myc mRNA and protein.

EN4 is a covalent ligand that targets cysteine 171 (C171) of MYC and is selective for c-MYC over N-MYC and L-MYC. EN4 inhibits MYC transcriptional activity, downregulates MYC targets, and impairs tumorigenesis.

APTO-253 (a.k.a. LOR-253, LT-253) inhibits c-Myc expression and selectively induces CDKN1A (p21), promotes G0-G1 cell-cycle arrest, and triggers apoptosis in acute myeloid leukemia (AML) cells. APTO-253 is also an inducer of KLF4 (Kruppel-like factor 4).

ML327 is an isoxazole compound that blocks MYC expression and tumor formation in neuroblastoma. ML327 also restores E-cadherin expression and induces apoptosis.

Mycro 3 is a potent and selective inhibitor of c-Myc in whole cell assays. Mycro 3 also shows weak inhibitory activity against AP-1.

KJ Pyr 9 is an inhibitor of MYC which interferes with MYC-MAX complex formation.

SaJM589 disrupts the Myc-Max heterodimer in a dose dependent manner.

KSI-3716 is a potent c-Myc inhibitor that blocks c-MYC/MAX binding to target gene promoters. KSI-3716 is an effective intravesical chemotherapy agent for bladder cancer.

KI-MS2-008 is a small molecule Max binder that modulates Myc-driven transcription in cells and inhibits viable cell levels in a Myc-dependent manner.

Triptolide (TPL) is a natural small molecule that has been shown to induce rapid reduction of c-Myc protein via a proteasome-dependent mechanism. its clinical use is largely limited due to poor aqueous solubility and dose-limited toxicity.

WBC100 (a.k.a., 14-dextrorotary-valyl-triptolide) is a water-soluble analog of TPL that selectively induces c-Myc protein degradation through ubiquitin E3 ligase CHIP mediated 26S proteasome pathway and potently regresses established c-Myc overexpressing tumors with good tolerability in multiple xenograft mouse models.

Other compounds can serve as MYC inhibitors in the present methods by affecting targets which inhibit MYC indirectly. Examples of these targets, and compounds which target them are shown below.

Aurora A Kinase: Aurora A Kinase binds to and stabilizes N-Myc, a Myc oncoprotein. Therefore, the inhibition of Aurora A kinase using small molecule inhibitors can be implemented as a strategy to target MYC function indirectly. Examples of these Aurora A kinase small molecule inhibitors include but are not limited to MLN8237 (a.k.a. alisertib), which renders the Aurora A-c-MYC/N-MYC complex dysfunctional, and CD532 which is a potent Aurora A kinase inhibitor that has the dual effect of blocking Aurora A kinase activity and driving degradation of MYCN. CD532 can destabilize N-Myc by disrupting the complex between Aurora-A and N-Myc.

mTOR inhibitors including Everolimus, Paxalisib, ATG-008, Gedatolisib, Samotolisib, Sapanisertib, and Apitolisib inhibit MYC protein biosynthesis.

eiF4A inhibitors including Zotatifin.

PLK1 (Polo-Like Kinase-1) directly binds and phosphorylates FBXW7 promoting its autopolyubiquitination and degradation resulting in stabilization of N-MYC. Onvansertib, CYC-140, and BI-6727 (a.k.a. Volasertib), are ATP-competitive PLK1 inhibitors that has been shown to inhibit MYC expression.

BRD4 (Bromodomain-containing protein 4) is a member of the bromodomain and extraterminal domain (BET) family of epigenetic readers. BRD4 is a transcriptional modulator of the MYC and MYCN genes. BRD4 directly interacts, phosphorylates and destabilizes MYC at the protein level through the formation of a ternary complex with ERK1 creating a regulatory network that balances MYC activity. Small compounds including JQ1, OTX015 (a.k.a. MK-8628, GSK2820151, ZEN-3694, CPI-0610 (a.k.a. Pelabresib), GSK525762 (a.k.a. I-BET762), BMS-986158, and INCB057643 have been used to therapeutically target BRD4/BET. Their safety, pharmacokinetics and activity are currently under assessment in Phase I and Phase I/II clinical trials including several cancers. dBET6 is a highly cell-permeable PROTAC degrader of BET bromodomains for BRD4 binding. dBET6 also induces c-MYC downregulation and apoptosis. Alobresib (a.k.a. GS-5829) is a novel BET inhibitor that represents a highly effective therapeutics agent against recurrent/chemotherapy-resistant USC-overexpressing c-Myc. Alobresib inhibits CLL cell proliferation and induces leukemia cell apoptosis through deregulation of key signaling pathways, such as BLK, AKT, ERK1/2, and MYC. Alobresib also inhibits NF-xB signaling (in phase 2 clinical trial). Inobrodib (a.k.a. CCS-1477, CBP-IN-1, and CBP/p300-IN-4) is a potent and selective inhibitor of p300/CBP bromodomain. Inobrodib works by inhibiting the expression and function of the androgen receptor (AR), as well as inhibiting c-Myc. BI-2536 is a dual Plk1 and BRD4 inhibitor and potently suppresses c-Myc expression. BI-2536 induces apoptosis and attenuates autophagy (in Phase 2 clinical trial).

PP2A (protein phosphatase 2) plays critical role in the SCF^{F}BXW⁷-mediated degradation of MYC. Therefore, PP2A activators such as LB-100, SMAP (DT-061), FTY720, OP449, and perphenazine can function as agents inhibiting MYC expression.

PIN1 (peptidyl-prolyl cis-trans isomerase NIMA-interacting 1) is essential in regulating MYC proteolysis via the Ub-proteasome system. PIN1 inhibitors such as KPT-6566, ATRA, BJP-06-005-3, and Sulfopin can function as agents for inhibiting MYC expression.

PIM1 (serine-threonine protein kinase pim-1) promotes T58 dephosphorylation and S62/S329 phosphorylation, stabilizing the MYC protein and increasing both its oncogenic transcriptional activity and transforming capacity. Therefore, PIM1 inhibitors such as AZD1208, TP-3654, PIM447, SGI-1776, LGH447, and SEL24 (a.k.a. MEN1703) can serve as inhibitors of MYC.

MRT-2359 is a highly selective molecular glue degrader of GSPT1, demonstrated substantial anticancer efficacy in MYC-driven tumors in preclinical setting and is currently being tested in the early phase clinical trial. GSPT1 is a small GTPase involved in catalyzing the termination phase of protein synthesis. MYC-driven cancers are addicted to protein translation, which creates a dependency on GSPT1.

Particular examples of heteroarotinoids include flexible heteroarotinoids having a urea or thiourea linker (referred to as “Flex-Hets”), such as, but not limited to any heteroarotinoid disclosed in U.S. Pat. Nos. 6,586,460 (see, for example, Columns 2-5 thereof) and 7,612,107 (see, for example, Columns 7-9 thereof). Non-limiting examples of heteroarotinoids that may be used herein include SHetA2, SHetA3, SHetA4, SHetC2, SHetD3, SHetD4, SHetD5, SHet50, SHet65, SHet100, OHet72, NHet17, NHet86, NHet90, and other heteroarotinoid compounds shown in Table 1, compounds 1-3 shown in Table 2, heteroarotinoid compounds shown in Table 3, heteroarotinoid compounds shown in Table 4, heteroarotinoid compounds shown in Table 5, heteroarotinoid compounds shown in Table 6, heteroarotinoid compounds shown in Table 7, and heteroarotinoid compounds shown in Table 8, as well as any combinations thereof.

TABLE 1
Heteroarotinoids (Benbrook et al., 1997*)

| | | | |
|---|---|---|---|
| 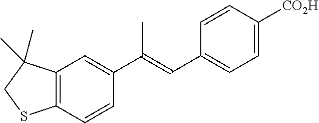 [SMILES: C/C(=C\c1ccc(C(=O)O)cc1)c1ccc2c(c1)C(C)(C)CS2] | 56 | 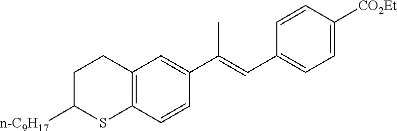 | 43 |
| 11 | | 16 | |
| 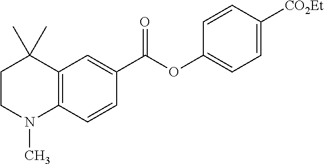 [SMILES: CCOC(=O)c1ccc(OC(=O)c2ccc3c(c2)C(C)(C)CCN3C)cc1] | 54 | 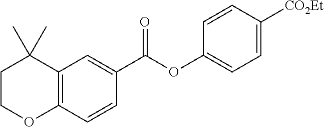 [SMILES: CCOC(=O)c1ccc(OC(=O)c2ccc3c(c2)C(C)(C)CCO3)cc1] | 40 |
| 19 | | 18 | |
| 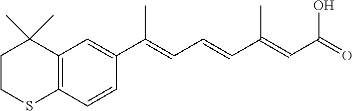 [SMILES: CC(/C=C/C=C(\C)c1ccc2c(c1)C(C)(C)CCS2)=C\C(=O)O] | 52 | 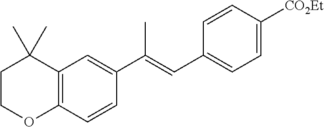 [SMILES: CCOC(=O)c1ccc(/C=C(\C)c2ccc3c(c2)C(C)(C)CCO3)cc1] | 35.7 |
| 8 | | 14 | |
| 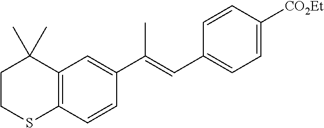 [SMILES: CCOC(=O)c1ccc(/C=C(\C)c2ccc3c(c2)C(C)(C)CCS3)cc1] | 51 | 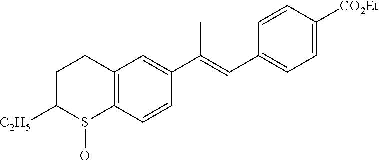 | 35.8 |
| 15 | | 17 | |
| 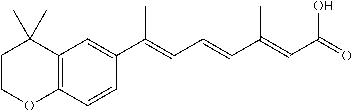 [SMILES: CC(/C=C/C=C(\C)c1ccc2c(c1)C(C)(C)CCO2)=C\C(=O)O] | 50 | 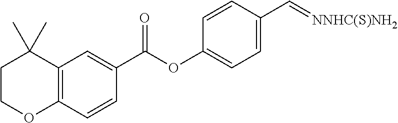 | 33 |
| 10 | | 20 | |
| 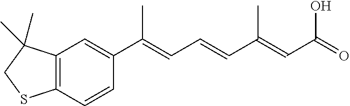 [SMILES: CC(/C=C/C=C(\C)c1ccc2c(c1)C(C)(C)CS2)=C\C(=O)O] | 50 | 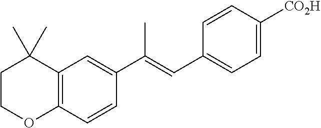 [SMILES: C/C(=C\c1ccc(C(=O)O)cc1)c1ccc2c(c1)C(C)(C)CCO2] | 34 |
| 12 | | 13 | |
| 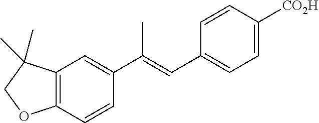 [SMILES: C/C(=C\c1ccc(C(=O)O)cc1)c1ccc2c(c1)C(C)(C)CO2] | 46 | 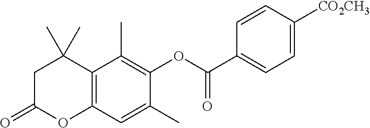 [SMILES: COC(=O)c1ccc(C(=O)Oc2c(C)cc3c(c2C)C(C)(C)CC(=O)O3)cc1] | 29 |
| 9 | | 21 | |

*D. M. Benbrook et al., “Biologically Active Heteroarotinoids Exhibiting Anticancer Activity and Decreased Toxicity,” J. Med. Chem., 1977, 40 (3567-3583).

TABLE 2
Nitrogen Heteroarotinoids (Dhar et al., 1999*)

| | | |
|---|---|---|
| 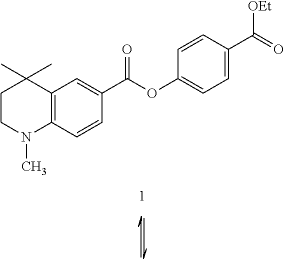 [SMILES: CCOC(=O)c1ccc(OC(=O)c2ccc3c(c2)C(C)(C)CCN3C)cc1] | 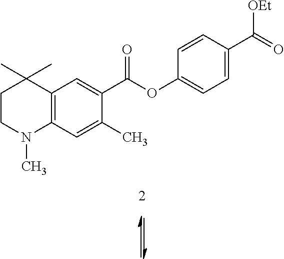 [SMILES: CCOC(=O)c1ccc(OC(=O)c2cc3c(cc2C)N(C)CCC3(C)C)cc1] | 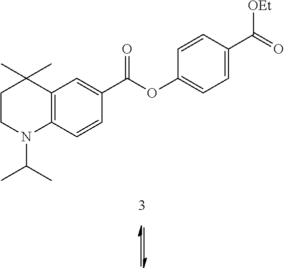 [SMILES: CCOC(=O)c1ccc(OC(=O)c2ccc3c(c2)C(C)(C)CCN3C(C)C)cc1] |
| 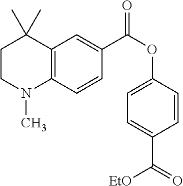 [SMILES: CCOC(=O)c1ccc(OC(=O)c2ccc3c(c2)C(C)(C)CCN3C)cc1] | 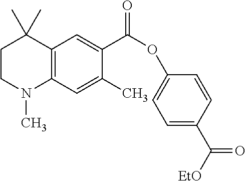 [SMILES: CCOC(=O)c1ccc(OC(=O)c2cc3c(cc2C)N(C)CCC3(C)C)cc1] | 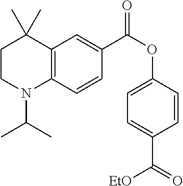 [SMILES: CCOC(=O)c1ccc(OC(=O)c2ccc3c(c2)C(C)(C)CCN3C(C)C)cc1] |
| 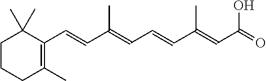 [SMILES: CC1=C(/C=C/C(C)=C/C=C/C(C)=C/C(=O)O)C(C)(C)CCC1] | 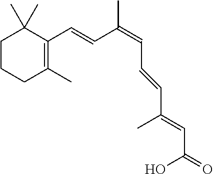 [SMILES: CC1=C(/C=C/C(C)=C\C=C\C(C)=C\C(=O)O)C(C)(C)CCC1] | 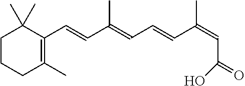 [SMILES: CC1=C(/C=C/C(C)=C/C=C/C(C)=C\C(=O)O)C(C)(C)CCC1] |
| t-RA | 9-c-RA | 13-c-RA |

| | |
|---|---|
| 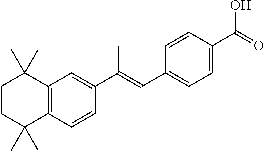 [SMILES: C/C(=C\c1ccc(C(=O)O)cc1)c1ccc2c(c1)C(C)(C)CCC2(C)C] | 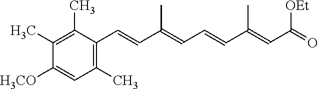 [SMILES: CCOC(=O)/C=C(C)/C=C/C=C(C)/C=C/c1c(C)cc(OC)c(C)c1C] |
| TTNBP | Etretinate |

*A. Dhar et al., “Synthesis, Structure-Activity Relationships, and RARy-Ligand Interactions of Nitrogen Heteroarotinoids,” J. Med. Chem., 1999, 42 (3602-3614).

TABLE 3
Heteroarotinoids (Zacheis et al., 1997*)

| | |
|---|---|
| 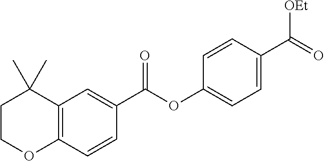 [SMILES: CCOC(=O)c1ccc(OC(=O)c2ccc3c(c2)C(C)(C)CCO3)cc1] | 5 |
| 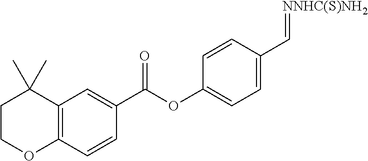 | 6 |
| 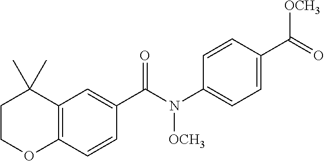 [SMILES: COC(=O)c1ccc(N(OC)C(=O)c2ccc3c(c2)C(C)(C)CCO3)cc1] | 7 |
| 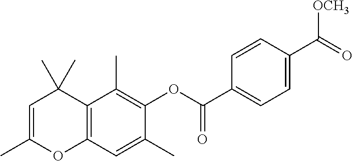 [SMILES: COC(=O)c1ccc(C(=O)Oc2c(C)cc3c(c2C)C(C)(C)C=C(C)O3)cc1] | 8 |
| 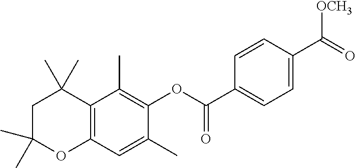 [SMILES: COC(=O)c1ccc(C(=O)Oc2c(C)cc3c(c2C)C(C)(C)CC(C)(C)O3)cc1] | 9 |
| 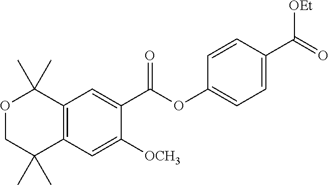 [SMILES: CCOC(=O)c1ccc(OC(=O)c2cc3c(cc2OC)C(C)(C)COC3(C)C)cc1] | 10 |
| 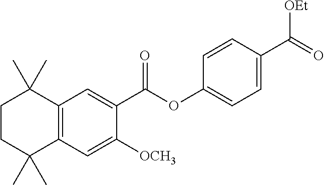 [SMILES: CCOC(=O)c1ccc(OC(=O)c2cc3c(cc2OC)C(C)(C)CCC3(C)C)cc1] | 11 |
| 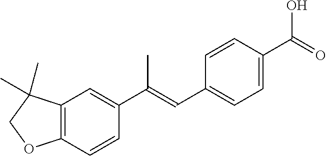 [SMILES: C/C(=C\c1ccc(C(=O)O)cc1)c1ccc2c(c1)C(C)(C)CO2] | 12 |
| 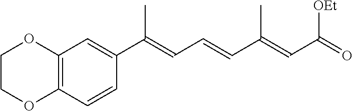 [SMILES: CCOC(=O)/C=C(C)/C=C/C=C(\C)c1ccc2c(c1)OCCO2] | 13 |
| 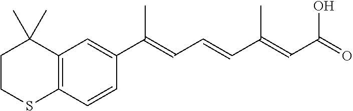 [SMILES: CC(/C=C/C=C(\C)c1ccc2c(c1)C(C)(C)CCS2)=C\C(=O)O] | 14 |
| 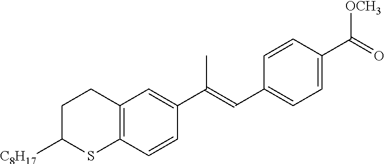 | 15 |
| 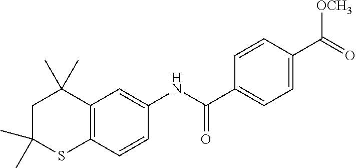 [SMILES: COC(=O)c1ccc(C(=O)Nc2ccc3c(c2)C(C)(C)CC(C)(C)S3)cc1] | 16 |
| 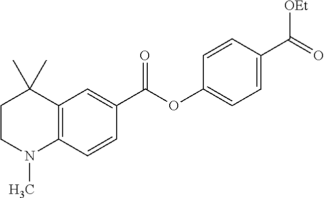 [SMILES: CCOC(=O)c1ccc(OC(=O)c2ccc3c(c2)C(C)(C)CCN3C)cc1] | 17 |
| 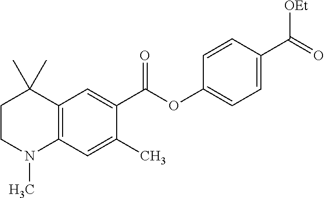 [SMILES: CCOC(=O)c1ccc(OC(=O)c2cc3c(cc2C)N(C)CCC3(C)C)cc1] | 18 |
| 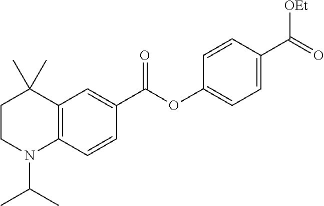 [SMILES: CCOC(=O)c1ccc(OC(=O)c2ccc3c(c2)C(C)(C)CCN3C(C)C)cc1] | 19 |

*D. Zacheis et al., “Heteroarotinoids Inhibit Head and Neck Cancer Cell Lines in Vitro and in Vivo Through Both RAR and RXR Retinoic Acid Receptors,” J. Med. Chem., 1999, 42 (4434-4445).

TABLE 4
Heteroarotinoids (Brown et al., 2004*)

| | | | |
|---|---|---|---|
| 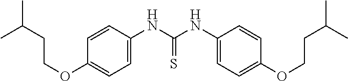 [SMILES: CC(C)CCOc1ccc(NC(=S)Nc2ccc(OCCC(C)C)cc2)cc1] | 0.5-1.0 | 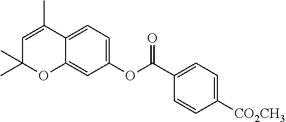 [SMILES: COC(=O)c1ccc(C(=O)Oc2ccc3c(c2)OC(C)(C)C=C3C)cc1] | 2.0-4.0 |
| 7 [Isoxy] | | 8 | |
| 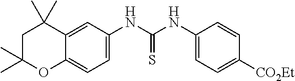 [SMILES: CCOC(=O)c1ccc(NC(=S)Nc2ccc3c(c2)C(C)(C)CC(C)(C)O3)cc1] | 5.0-10.0 | 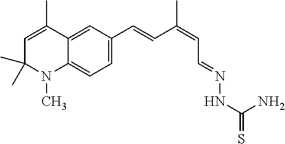 [SMILES: CC1=CC(C)(C)N(C)c2ccc(/C=C/C(C)=C\C=N\NC(N)=S)cc21] | 10.0-20.0 |
| 9 | | 10 | |
| 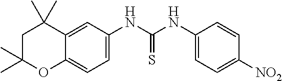 [SMILES: CC1(C)CC(C)(C)c2cc(NC(=S)Nc3ccc([N+](=O)[O-])cc3)ccc2O1] | 20.0-40.0 | 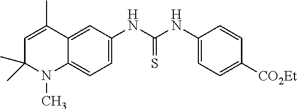 [SMILES: CCOC(=O)c1ccc(NC(=S)Nc2ccc3c(c2)C(C)=CC(C)(C)N3C)cc1] | 20.0-40.0 |
| 11 | | 12 | |
| 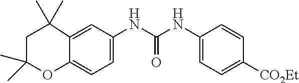 [SMILES: CCOC(=O)c1ccc(NC(=O)Nc2ccc3c(c2)C(C)(C)CC(C)(C)O3)cc1] | 20.0-40.0 | 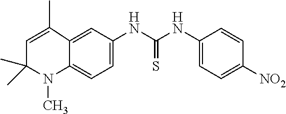 [SMILES: CC1=CC(C)(C)N(C)c2ccc(NC(=S)Nc3ccc([N+](=O)[O-])cc3)cc21] | 20.0-40.0 |
| 13 | | 14 | |
| 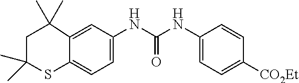 [SMILES: CCOC(=O)c1ccc(NC(=O)Nc2ccc3c(c2)C(C)(C)CC(C)(C)S3)cc1] | 20.0-40.0 | 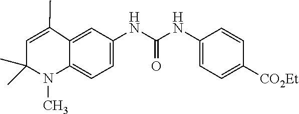 [SMILES: CCOC(=O)c1ccc(NC(=O)Nc2ccc3c(c2)C(C)=CC(C)(C)N3C)cc1] | 20.0-40.0 |
| 15 | | 16 | |
| 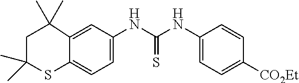 [SMILES: CCOC(=O)c1ccc(NC(=S)Nc2ccc3c(c2)C(C)(C)CC(C)(C)S3)cc1] | 20.0-40.0 | 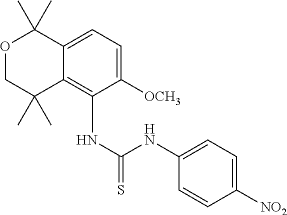 [SMILES: COc1ccc2c(c1NC(=S)Nc1ccc([N+](=O)[O-])cc1)C(C)(C)COC2(C)C] | 20.0-40.0 |
| 17 | | 18 | |
| 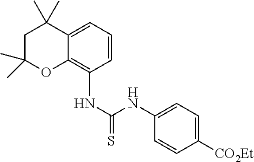 [SMILES: CCOC(=O)c1ccc(NC(=S)Nc2cccc3c2OC(C)(C)CC3(C)C)cc1] | 20.0-40.0 | 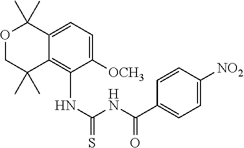 [SMILES: COc1ccc2c(c1NC(=S)NC(=O)c1ccc([N+](=O)[O-])cc1)C(C)(C)COC2(C)C] | 20.0-40.0 |
| 19 | | 20 | |
| 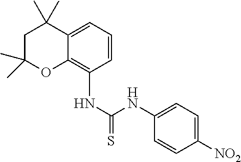 [SMILES: CC1(C)CC(C)(C)c2cccc(NC(=S)Nc3ccc([N+](=O)[O-])cc3)c2O1] | 20.0-40.0 | 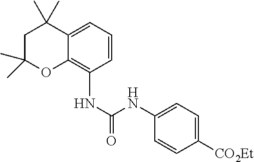 [SMILES: CCOC(=O)c1ccc(NC(=O)Nc2cccc3c2OC(C)(C)CC3(C)C)cc1] | 20.0-40.0 |
| 21 | | 22 | |

*C. W. Brown et al., “Novel Heteroarotinoids as Potential Antagonists of Mycobacterium bovisBCG,” J. Med. Chem., 2004, 47 (1008-1017).

TABLE 5
Heteroarotinoids (Le et al., 2007*)

| | |
|---|---|
| 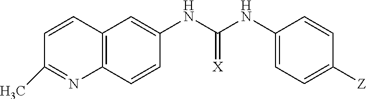 | 9 |
| a. X = S; Z = NO₂ | |
| b. X = O; Z = NO₂ | |
| c. X = O; Z = CO₂Et | |
| 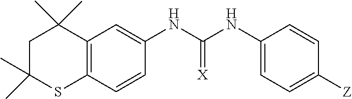 | 10 |
| a. X = S; Z = NO₂ | |
| b. X = S; Z = CO₂Et | |
| c. X = O; Z = NO₂ | |
| d. X = O; Z = CO₂Et | |

*T.C. Le et al., “Heteroarotinoids with Anti-Cancer Activity Against Ovarian Cancer Cells,” TheOpen Medicinal Chemistry Journal, 2007, 1 (11-23).

TABLE 6
Heteroarotinoids (Liu et al., 2004*)

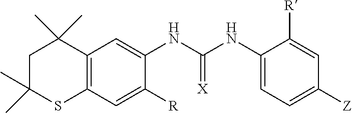

15a-h

| entry | R | X | R | Z | product | yield (%) |
|---|---|---|---|---|---|---|
| 1 | H | O | H | CO₂Et | 15a | 64 |
| 2 | H | S | H | CO₂Et | 15b | 92 |
| 3 | H | S | H | NO₂ | 15c | 83 |
| 4 | H | S | H | SO₂NH₂ | 15d | 87 |
| 5 | H | S | CH₃ | NO₂ | 15e | 82 |
| 6 | CH₃ | O | H | CO₂Et | 15f | 43 |
| 7 | CH₃ | S | H | CO₂Et | 15g | 66 |
| 8 | CH₃ | S | H | SO₂NH₂ | 15h | 56 |

*S. Liu et al., “Synthesis of Flexible Sulfur-Containing Heteroarotinoids That Induce Apoptosis and Reactive Oxygen Species with Discrimination between Malignant and Benign Cells,” J. Med. Chem., 2004, 47 (999-1007).

TABLE 7
Heteroarotinoids (Gnanasekaran et al., 2015*)

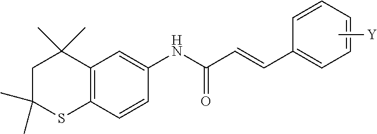

18a-h
20a-c

| | |
|---|---|
| Compound | Y |

Nitrogen-Substituted Flex-Hets

| | |
|---|---|
| 5 (SHetA2) | 4-NO₂ |
| 18b | 4-NO₂ |
| 18f | 4-N(CH₃)₂ |
| 18h | 4-NH₂ |
| 20a | 4-N-methylamide |
| 20b | 4-N-cyclopropylamide |
| 20c | 4-oxomorpholino |

Non-Nitrogen-Substituted Flex-Hets

| | |
|---|---|
| 18a | 4-CO₂Et |
| 18c | 4-CF₃ |
| 18d | 2,3,4,5,6-F |
| 18e | 3,4-OCH₃ |
| 18g | 4-CO₂H |

*K. K. Gnanasekaran et al., “Synthesis and evaluation of second generation Flex-Het scaffoldsagainst the human ovarian A2780 cancer cell line,” European J. Med. Chem., 2015, 96 (209-217).

TABLE 8
Heteroarotinoids (Liu et al., 2009*)
SHetC2

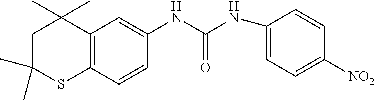 [SMILES: CC1(C)CC(C)(C)c2cc(NC(=O)Nc3ccc([N+](=O)[O-])cc3)ccc2S1]

SHetA2

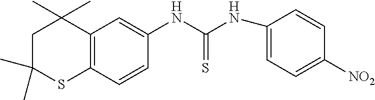 [SMILES: CC1(C)CC(C)(C)c2cc(NC(=S)Nc3ccc([N+](=O)[O-])cc3)ccc2S1]

SHetA4

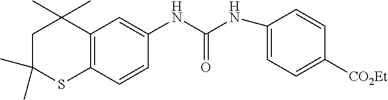 [SMILES: CCOC(=O)c1ccc(NC(=O)Nc2ccc3c(c2)C(C)(C)CC(C)(C)S3)cc1]

SHetA3

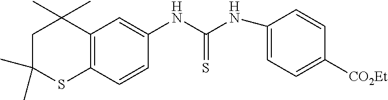 [SMILES: CCOC(=O)c1ccc(NC(=S)Nc2ccc3c(c2)C(C)(C)CC(C)(C)S3)cc1]

SHetD3

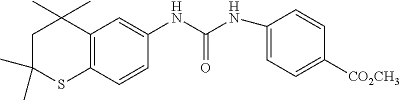 [SMILES: COC(=O)c1ccc(NC(=O)Nc2ccc3c(c2)C(C)(C)CC(C)(C)S3)cc1]

SHetD4

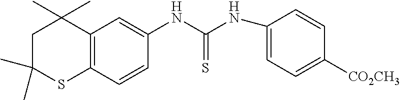 [SMILES: COC(=O)c1ccc(NC(=S)Nc2ccc3c(c2)C(C)(C)CC(C)(C)S3)cc1]

SHetD5

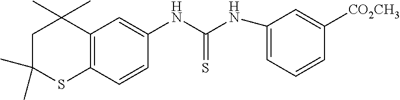 [SMILES: COC(=O)c1cccc(NC(=S)Nc2ccc3c(c2)C(C)(C)CC(C)(C)S3)c1]

*T. Liu et al., “Development of flexible-heteroarotinoids for kidney cancer,” Mol. Cancer Ther., 2009, 8(5), (1227-1238).

“Treatment” refers to therapeutic treatments. “Prevention” refers to prophylactic or preventative treatment measures or reducing the onset of a condition or disease. The term “treating” refers to administering the composition to a subject for therapeutic purposes and/or for prevention. Non-limiting examples of modes of administration include inhalation, oral, topical, retrobulbar, subconjunctival, transdermal, parenteral, subcutaneous, intranasal, intramuscular, intraperitoneal, intravitreal, and intravenous routes, including both local and systemic applications. In addition, the compositions of the present disclosure may be designed to provide delayed, controlled, extended, and/or sustained release using formulation techniques which are well known in the art.

The terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art.

Where used herein the term “drug combination” refers to a combination of drugs (compounds) which can be conjointly administered.

As used herein the terms “conjoint,” “conjointly,” “conjointly administered,” or “conjoint administration” refers to any form of administration of a combination of two or more different therapeutic compounds (also referred to herein as drugs or active agents) such that the second compound is administered while the previously administered therapeutic compound is still effective in the body, whereby the two or more compounds are simultaneously active in the patient, enabling a synergistic interaction of the compounds. For example, the different therapeutic compounds can be administered either together in the same formulation (i.e., as a physical mixture), or in separate formulations, either concomitantly (at the same time) or sequentially. When administered sequentially the different compounds may be administered immediately in succession, or separated by a suitable duration of time, as long as the active agents function together in a synergistic manner.

The term “effective amount” refers to an amount of an active agent which is sufficient to exhibit a detectable therapeutic effect without excessive adverse side effects (such as toxicity, irritation and allergic response) commensurate with a reasonable benefit/risk ratio when used in the manner disclosed herein. The effective amount for a patient will depend upon the type of patient, the patient's size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

The term “ameliorate” means a detectable or measurable improvement in a subject's condition, disease or symptom thereof. A detectable or measurable improvement includes a subjective or objective decrease, reduction, inhibition, suppression, limit or control in the occurrence, frequency, severity, progression, or duration of the condition or disease, or an improvement in a symptom or an underlying cause or a consequence of the disease, or a reversal of the disease. A successful treatment outcome can lead to a “therapeutic effect,” or “benefit” of ameliorating, decreasing, reducing, inhibiting, suppressing, limiting, controlling or preventing the occurrence, frequency, severity, progression, or duration of a disease or condition, or consequences of the disease or condition in a subject.

A decrease or reduction in worsening, such as stabilizing the condition or disease, is also a successful treatment outcome. A therapeutic benefit therefore need not be complete ablation or reversal of the disease or condition, or any one, most or all adverse symptoms, complications, consequences or underlying causes associated with the disease or condition. Thus, a satisfactory endpoint may be achieved when there is an incremental improvement such as a partial decrease, reduction, inhibition, suppression, limit, control, or prevention in the occurrence, frequency, severity, progression, or duration, or inhibition or reversal of the condition or disease (e.g., stabilizing), over a short or long duration of time (hours, days, weeks, months, etc.). Effectiveness of a method or use, such as a treatment that provides a potential therapeutic benefit or improvement of a condition or disease, can be ascertained by various methods and testing assays.

Where used herein the term cancer, cancer cell, or tumor, can be used in reference to various cancers including, but not limited to ovarian cancer, cervical cancer, peritoneal cancer, uterine cancer, vulvar cancer, oral cancer, pharyngeal cancer, oropharyngeal cancer, nasopharyngeal cancer, respiratory cancer, urogenital cancer, gastrointestinal cancer, central or peripheral nervous system tissue cancer, an endocrine cancer, neuroendocrine cancer, glioma, carcinoma, lymphoma, melanoma, fibroma, meningioma, brain cancer, renal cancer, pheochromocytoma, pancreatic islet cell cancer, Li-Fraumeni tumors, thyroid cancer, parathyroid cancer, pituitary tumors, adrenal gland tumors, osteogenic sarcoma tumors, multiple neuroendocrine type I and type II tumors, head and neck cancer, prostate cancer, esophageal cancer, tracheal cancer, liver cancer, bladder cancer, stomach cancer, pancreatic cancer, uterine cancer, testicular cancer, colon cancer, rectal cancer, and skin cancer, and any other cancer which comprises one or more mutations in the p53 gene.

In other embodiments, the term cancer, cancer cell, or tumor, can be used in reference to various cancers including, but not limited to breast cancer (e.g., estrogen receptor positive or negative, progesterone receptor positive or negative, HER-2 positive or negative, triple-negative breast cancer (TNBC), or BRCA1 and/or BRCA2 positive or negative), lung cancer (e.g., non-small cell lung cancer and small cell lung cancer), endometrial cancer, glioblastoma, B-cell malignancies, biliary tract cancer, bone cancer, choriocarcinoma, connective tissue cancer, cancers of the digestive system, gallbladder cancer, hepatocellular carcinoma, intra-epithelial neoplasm, kidney cancer, liver cancer, lymphoma, skin cancer (e.g., melanoma and basal cell carcinoma), neuroblastoma, mesothelioma, neuroglioma, oral cavity cancer, pediatric cancer, pancreatic endocrine tumors, pituitary adenoma, thymoma, renal cell carcinoma, salivary gland cancer, sarcoma (e.g., Ewing's sarcoma, fibrosarcoma, and rhabdomyosarcoma), small bowel cancer, ureteral cancer, cancers of the urinary system, and hematological cancers (e.g., acute myeloid leukemia and multiple myeloma).

Exemplary solid cancerous tumors that can be treated with the active agents of the present disclosure include, but are not limited to, a tumor of an organ selected from the group consisting of pancreas, colon, cecum, stomach, brain, liver, gallbladder, head, neck, ovary, kidney, larynx, sarcoma, lung, bladder, uterus, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AIL); and chronic myeloblastic leukemia.

The compounds of the present disclosure may be combined with one or more pharmaceutically-acceptable excipients, including carriers, vehicles, diluents, and adjuvants which may improve solubility, deliverability, dispersion, stability, and/or conformational integrity of the compounds or conjugates thereof.

“Pharmaceutically acceptable salts” means salts of active agent compounds disclosed herein which are pharmaceutically acceptable, as defined above, and which possess the desired pharmacological activity. Such salts include (but are not limited to) acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hyIdroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include (but are not limited to) base addition salts which may be formed when acidic protons present are capable of reacting with inorganic or organic bases. Acceptable inorganic bases include (but are not limited to) sodium hydroxide, sodium carbonate, potassium hydroxide, aluminum hydroxide and calcium hydroxide. Acceptable organic bases include (but are not limited to) ethanolamine, diethanolamine, trietbanolamine, tromethamine, N-methylglucamine and the like. It should be recognized that the particular anion or cation forming a part of any salt of the present disclosure is not critical, so long as the salt, as a whole, is pharmacologically acceptable Additional non-limiting examples of pharmaceutically acceptable salts and their methods of preparation and use are shown in Handbook of Pharmaceutical Salts: Properties, and Use (P. B. Stahl & C. G Wermuth eds., Verlag Helvetica Chimica Acta, 2002).

The term “coadministration” refers to administration of two or more active agents, e.g., at least one heteroarotinoid and at least one MYC inhibitor. The timing of coadministration depends in part on the combination and compositions administered and can include administration at the same time, just prior to, or just after the administration of one or more additional therapies. “Coadministration” is meant to include simultaneous or sequential administration of the compound and/or composition individually or in combination. Thus, the preparations can also be combined, when desired, with other active substances (e.g., to reduce metabolic degradation). For example, the compositions described herein can be used in combination with one another, or with other active agents known to be useful in treating MI, and co-occurring conditions thereof.

The active agents of the present disclosure may be present in the pharmaceutical compositions (alone or in combination) at any concentration that allows the pharmaceutical composition to function in accordance with the present disclosure; for example, but not by way of limitation, the compound(s) may be present in a carrier, diluent, or buffer solution in a wt/wt or vol/vol range having a lower level selected from 0.00001%, 0.0001%, 0.005%, 0.001%, 0.005%, 0.01%, 0.05%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9% and 2.0%; and an upper level selected from 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, and 95%. Non-limiting examples of particular wt/wt or vol/vol ranges include a range of from about 0.0001% to about 95%, a range of from about 0.001% to about 75%; a range of from about 0.005% to about 50%; a range of from about 0.01% to about 40%; a range of from about 0.05% to about 35%; a range of from about 0.1% to about 30%; a range of from about 0.1% to about 25%; a range of from about 0.1% to about 20%; a range of from about 1% to about 15%; a range of from about 2% to about 12%; a range of from about 5% to about 10%; and the like. Any other range that includes a lower level selected from the above-listed lower level concentrations and an upper level selected from the above-listed upper level concentrations also falls within the scope of the present disclosure. Percentages used herein may be weight percentages (wt %) or volume percentages (vol %).

In certain non-limiting embodiments, an effective amount or therapeutic dosage of a pharmaceutical composition of the present disclosure contains, sufficient active agent to deliver from about 0.001 μg/kg to about 100 mg/kg (weight of active agent/body weight of the subject). For example, the composition will deliver about 0.01 μg/kg to about 50 mg/kg, and more particularly about 0.1 μg/kg to about 10 mg/kg, and more particularly about 1 μg/kg to about 1 mg/kg. Practice of a method of the present disclosure may comprise administering to a subject an effective amount of the active agent in any suitable systemic and/or local formulation, in an amount effective to deliver the therapeutic dosage of the active agent. In certain embodiments, an effective dosage may be, in a range of about 1 μg/kg to about 1 mg/kg of the active agent.

In certain non-limiting embodiments, an effective amount or therapeutic dosage of a pharmaceutical composition of the present disclosure contains at least one heteroarotinoid (e.g., SHetA2) in a dose range that will result in 0.01 micromolar to 100 millimolar blood or tissue levels, and at least one c-MYC inhibitor in a concentration in a dose range that will result in 0.01 micromolar to 100 millimolar blood or tissue levels. In certain non-limiting embodiments, an effective amount or therapeutic dosage of a pharmaceutical composition of the present disclosure contains SHetA2 in a concentration in a dose range that will result in 0.01 micromolar to 100 millimolar, and a c-MYC inhibitor in a dose range that will result in 0.01 micromolar to 100 millimolar blood or tissue levels.

In non-limiting embodiments, the synergistic ratio of the compositions of the first active agent (heteroarotinoid) and the c-MYC inhibitor may be in a range from about 50:1 wt/wt to about 1:50 wt/wt, about 45:1 wt/wt to about 1:45 wt/wt, about 40:1 wt/wt to about 1:40 wt/wt, about 35:1 wt/wt to about 1:35 wt/wt, about 30:1 wt/wt to about 1:30 wt/wt, about 25:1 wt/wt to about 1:25 wt/wt, e.g., 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, or 1:25 or may be in any range bounded by the ratios listed above.

The term “synergistic” or “synergistic effect” or “synergistic interaction” as used herein refers to a therapeutic combination which is more effective than the additive effects of the two or more single active agents. A “synergistic ratio” is a ratio of two compounds which results in a synergistic effect. A determination of a synergistic interaction between the active agents described herein may be based on the results obtained from the assays described herein. The results of these assays can be analyzed by using, in one non-limiting method, the Chou and Talalay combination method (Chou T C, Talalay P. “Quantitative analysis of dose-effect relationships: the combined effects of multiple drugs or enzyme inhibitors.” Adv Enzyme Regul. 1984; 22: 27-55) and Dose-Effect Analysis with CalcuSyn software in order to obtain a Combination Index. The combinations provided herein have been evaluated in several assay systems, and the data can be analyzed utilizing a standard program for quantifying synergism, additivism, and antagonism among anticancer agents. An example program is that described by Chou and Talalay, in “New Avenues in Developmental Cancer Chemotherapy,” Academic Press, 1987, Chapter 2. Combination Index values less than 0.9 indicate synergy, values greater than 1.2 indicate antagonism and values between 0.9 to 1.1 indicate additive effects (e.g., see Table 9 below).

TABLE 9
Interpretation of Combination Index (CI)

| CI Range | Interpretation |
|---|---|
| <0.1 | Very Strong Synergism |
| 0.1-0.3 | Strong Synergism |
| 0.3-0.7 | Synergism |
| 0.7-0.85 | Moderate Synergism |
| 0.85-0.90 | Slight Synergism |
| 0.90-1.10 | Nearly Additive |
| 1.10-1.20 | Slight Antagonism |
| 1.20-1.45 | Moderate Antagonism |
| 1.45-3.3 | Antagonism |
| 3.3-10 | Strong Antagonism |

Another non-limiting method which may be used to demonstrate a synergistic interaction is the Zero Interaction Potency (ZIP) synergy model, in which a score >0 indicates a synergistic response between the two drugs (Yadav, B. et al. Searching for drug synergy in complex dose-response landscapes using an interaction potency model. Computational and Structural Biotechnology Journal 13, 504-513 (2015)).

The combination therapy may provide “synergy” and prove “synergistic”, i.e., the effect achieved when the active agents used together is greater than the sum of the effects that results from using the compounds separately. A synergistic effect may be attained when the active agents are: (1) co-formulated and administered or delivered simultaneously in a combined, unit dosage formulation; (2) delivered in succession (“alternation therapy”) or in parallel as separate formulations; or (3) by some other effective regimen. When delivered in successive administrations, a synergistic effect may be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

Certain non-limiting embodiments of the present disclosure are directed to a method that comprises administering to a subject in need thereof any of the pharmaceutical compositions disclosed or otherwise contemplated herein.

The pharmaceutical compositions may be administered via any mechanisms disclosed herein or otherwise contemplatable by a person having ordinary skill in the art. In one non-limiting embodiment, the administration occurs via an inhaler, which aerosolizes the active agents.

The pharmaceutical compositions of the present disclosure may be administered for any purpose disclosed or otherwise contemplated herein, as well as for any purpose within the purview of a person having ordinary skill in the art. In one non-limiting embodiment, the pharmaceutical compositions are administered in a method of treating or reducing the occurrence of cancer. However, this treatment method is not to be construed as limiting of the present disclosure, and any diseases, disorders, or conditions disclosed herein or otherwise contemplatable by a person having ordinary skill in the art (given the subject application) which may derive a therapeutic effect by treatment with the compositions disclosed herein also fall within the scope of the methods of the present disclosure.

Practice of the methods of the present disclosure may comprise administering to a subject therapeutically effective amounts of the active agents in any suitable systemic and/or local formulation, in an amount effective to deliver the dosages listed herein. The dosage can be administered, for example but not by way of limitation, on a one-time basis, or administered at multiple times (for example but not by way of limitation, from one to five times per day, or once or twice per week), or continuously via a venous drip, depending on the desired therapeutic effect. In one non-limiting example of a therapeutic method of the present disclosure, the active agent is provided in an IV infusion in the range of from about 0.01 mg/kg to about 10 mg/kg of body weight once a day.

The compositions and dosage forms of the present disclosure can be administered in a single dose treatment or in multiple dose treatments on a schedule and over a time period appropriate to the age, weight, and condition of the subject, the particular composition used, and the route of administration. In one non-limiting embodiment, a single dose of the composition according to the disclosure is administered. In other non-limiting embodiments, multiple doses are administered. The frequency of administration can vary depending on any of a variety of factors, e.g., severity of the symptoms, or whether the composition is used for prophylactic or curative purposes. For example, in certain non-limiting embodiments, the composition is administered once per month, twice per month, three times per month, every other week, once per week, twice per week, three times per week, four times per week, five times per week, six times per week, every other day, daily, twice a day, or three times a day. The duration of treatment (i.e., the period of time over which the composition is administered) can vary, depending on any of a variety of factors, e.g., subject response. For example, the composition can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. Where used herein, unless otherwise indicated, the dosage amount refers to the amount of active pharmaceutical ingredient (API) that is administered to the subject.

The dosage of an administered active agent for humans will vary depending upon factors such as (but not limited to) the patient's age, weight, height, sex, general medical condition, and previous medical history. In certain non-limiting embodiments, where the active agent is administered by injection or infusion, the recipient may be provided with a dosage of the active agent that is in the range of from about 1 mg to about 1000 mg, and it may be administered as a single infusion or multiple injections, although a lower or higher dosage also may be administered. In certain non-limiting embodiments, the dosage may be in the range of from about 25 mg to about 100 mg of the active agent per square meter (m²) of body surface area for a typical adult, although a lower or higher dosage also may be administered. Non-limiting examples of dosages of the active agent that may be administered to a human subject include, but are not limited to, those in ranges of about 1 to about 1000 mg, about 1 to about 600 mg, about 1 to about 500 mg, about 1 to about 400 mg, about 1 to about 300 mg, about 1 to about 200 mg, about 100 to about 600 mg, about 100 to about 500 mg, about 100 to about 400 mg, about 100 to about 300 mg, about 100 to about 200 mg, about 150 to about 600 mg, about 150 to about 500 mg, about 150 to about 400 mg, about 150 to about 300 mg, about 150 to about 250 mg, about 150 to about 200 mg, about 200 to about 7500 mg, about 200 to about 600 mg, about 200 to about 500 mg, about 200 to about 400 mg, about 200 to about 300 mg, and about 200 to about 250 mg, or any subrange within any of the aforementioned ranges. Dosages may be repeated as needed, for example (but not by way of limitation), once per week for 4-10 weeks, once per week for 8 weeks, or once per week for 4 weeks. It may also be given less frequently, such as (but not limited to) every other week for several months, or more frequently, such as twice weekly or by continuous infusion.

In certain non-limiting embodiments, the present disclosure is directed to a dosing regimen comprising multiple dosing cycles (e.g., wherein the first dosing cycle is a step-up, fractionated dosing cycle). In some non-limiting embodiments, the dose may range from about 50 mg to about 200 mg (e.g., from about 50 mg to about 175 mg, from about 50 mg to about 150 mg, from about 50 mg to about 125 mg, from about 50 mg to about 100 mg, from about 50 mg to about 75 mg, from about 50 mg to about 70 mg, from about 52 mg to about 100 mg, from about 52 mg to about 75 mg, from about 50 mg to about 180 mg, from about 55 mg to about 150 mg, from about 55 mg to about 100 mg, from about 55 mg to about 70 mg, from about 55 mg to about 65 mg, from about 58 mg to about 62 mg; e.g., about about 60 mg, or any subrange within any of the aforementioned ranges). In some non-limiting embodiments, the dose may be about 60 mg. In some non-limiting embodiments, the dose is about 1 mg. In some non-limiting embodiments, the dose is about 2 mg.

In some non-limiting embodiments, the dose is from about 20 mg to about 200 mg (e.g., from about 20 mg to about 175 mg, from about 20 mg to about 150 mg, from about 20 mg to about 100 mg, from about 20 mg to about 75 mg, from about 30 mg to about 175 mg, from about 40 mg to about 175 mg, from about 45 mg to about 175 mg, from about 50 mg to about 175 mg, from about 30 mg to about 150 mg, from about 40 mg to about 100 mg, from about 45 mg to about 75 mg, from about 50 mg to about 70 mg, from about 55 mg to about 65 mg, from about 58 mg to about 62 mg; about 20 mg, about 30 mg, about 45 mg, or e.g., about 60 mg, or any subrange within any of the aforementioned ranges). In some non-limiting embodiments, the dose is from about 12 mg to about 48 mg (e.g., from about 12 mg to about 42 mg, from about 12 mg to about 36 mg, from about 12 mg to about 30 mg, from about 18 mg to about 48 mg, from about 18 mg to about 42 mg, from about 24 mg to about 42 mg, from about 27 mg to about 42 mg, from about 24 mg to about 36 mg, from about 27 mg to about 33 mg, from about 28 mg to about 32 mg; e.g., about 24 mg, about 27 mg, about 30 mg, about 33 mg, or about 36 mg, or any subrange within any of the aforementioned ranges).

In some non-limiting embodiments, the dosing regimen comprises administration of a dose in a range of from about 100 mg to about 750 mg (e.g., from about 100 mg to about 725 mg, from about 100 mg to about 700 mg, from about 100 mg to about 675 mg, from about 100 mg to about 650 mg, from about 100 mg to about 625 mg, from about 100 mg to about 600 mg, from about 100 mg to about 575 mg, from about 100 mg to about 550 mg, from about 100 mg to about 525 mg, from about 100 mg to about 500 mg, from about 100 mg to about 475 mg, from about 100 mg to about 450 mg, from about 100 mg to about 425 mg, from about 100 mg to about 400 mg, from about 100 mg to about 375 mg, from about 100 mg to about 350 mg, from about 100 mg to about 325 mg, from about 100 mg to about 300 mg, from about 100 mg to about 275 mg, from about 100 mg to about 250 mg, or from about 100 mg to about 225 mg, from about 100 mg to about 200 mg, from about 100 mg to about 175 mg, from about 100 mg to about 150 mg, or from about 100 mg to about 125 mg, or any subrange within any of the aforementioned ranges).

In some non-limiting embodiments, the dosing regimen comprises administration of a dose in a range of from about 200 mg to about 750 mg (e.g., from about 200 mg to about 725 mg, from about 200 mg to about 700 mg, from about 200 mg to about 675 mg, from about 200 mg to about 650 mg, from about 200 mg to about 625 mg, from about 200 mg to about 600 mg, from about 200 mg to about 575 mg, from about 200 mg to about 550 mg, from about 200 mg to about 525 mg, from about 200 mg to about 500 mg, from about 200 mg to about 475 mg, from about 200 mg to about 450 mg, from about 200 mg to about 425 mg, from about 200 mg to about 400 mg, from about 200 mg to about 375 mg, from about 200 mg to about 350 mg, from about 200 mg to about 325 mg, from about 200 mg to about 300 mg, from about 200 mg to about 275 mg, from about 200 mg to about 250 mg, or from about 200 mg to about 225 mg, or any subrange within any of the aforementioned ranges).

In some non-limiting embodiments, the dosing regimen comprises administration of a dose in a range of from about 300 mg to about 750 mg (e.g., from about 300 mg to about 725 mg, from about 300 mg to about 700 mg, from about 300 mg to about 675 mg, from about 300 mg to about 650 mg, from about 300 mg to about 625 mg, from about 300 mg to about 600 mg, from about 300 mg to about 575 mg, from about 300 mg to about 550 mg, from about 300 mg to about 525 mg, from about 300 mg to about 500 mg, from about 300 mg to about 475 mg, from about 300 mg to about 450 mg, from about 300 mg to about 425 mg, from about 300 mg to about 400 mg, from about 300 mg to about 375 mg, from about 300 mg to about 350 mg, or from about 300 mg to about 325 mg, or any subrange within any of the aforementioned ranges).

In some non-limiting embodiments, the dosing regimen comprises administration of a dose in a range of from about 400 mg to about 750 mg (e.g., from about 400 mg to about 725 mg, from about 400 mg to about 700 mg, from about 400 mg to about 675 mg, from about 400 mg to about 650 mg, from about 400 mg to about 625 mg, from about 400 mg to about 600 mg, from about 400 mg to about 575 mg, from about 400 mg to about 550 mg, from about 400 mg to about 525 mg, from about 400 mg to about 500 mg, from about 400 mg to about 475 mg, from about 400 mg to about 450 mg, or from about 400 mg to about 425 mg, or any subrange within any of the aforementioned ranges).

In some non-limiting embodiments, the dosing regimen comprises administration of a dose in a range of from about 500 mg to about 750 mg (e.g., from about 500 mg to about 725 mg, from about 500 mg to about 700 mg, from about 500 mg to about 675 mg, from about 500 mg to about 650 mg, from about 500 mg to about 625 mg, from about 500 mg to about 600 mg, from about 500 mg to about 575 mg, from about 500 mg to about 550 mg, or from about 500 mg to about 525 mg, or any subrange within any of the aforementioned ranges).

In some non-limiting embodiments, the dosing regimen comprises administration of a dose in a range of from about 600 mg to about 750 mg (e.g., from about 600 mg to about 725 mg, from about 600 mg to about 700 mg, from about 600 mg to about 675 mg, from about 600 mg to about 650 mg, from about 600 mg to about 625 mg, or any subrange within any of the aforementioned ranges). In some non-limiting embodiments, the active agent is provided in a concentration of about 1 nM, about 5 nM, about 10 nM, about 25 nM, about 50 nM, about 75 nM, about 100 nM, about 150 nM, about 200 nM, about 250 nM, about 300 nM, about 350 nM, about 400 nM, about 500 nM, about 550 nM, about 600 nM, about 700 nM, about 800 nM, about 900 nM, about 1 μM, about 2 μM, about 3 μM, about 4 μM, about 5 μM, about 6 μM, about 7 μM, about 8 μM, about 9 μM, about 10 μM, about 15 μM, about 20 μM, about 25 μM, about 30 μM, about 35 μM, about 40 μM, about 45 μM, about 50 μM, about 60 μM, about 70 μM, about 75 μM, about 80 μM, about 90 μM, about 100 μM, about 125 μM, about 150 μM, about 175 μM, about 200 μM, about 250 μM, about 300 μM, about 350 μM, about 400 μM, about 500 μM, about 600 μM, about 700 μM, about 750 μM, about 800 μM, about 900 μM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 100 mM, about 110 mM, about 120 mM, about 130 mM, about 140 mM, about 150 mM, about 160 mM, about 170 mM, about 180 mM, about 190 mM, about 200 mM, about 250 mM, about 300 mM, about 400 mM, about 500 mM, about 600 mM, about 700 mM, about 800 mM, about 900 mM, about 1000 mM, about 1 M, about 1.1 M, about 1.2 M, about 1.3 M, about 1.4 M, about 1.5 M, about 1.6 M, about 1.7 M, about 1.8 M, about 1.9 M, about 2 M, about 3 M, about 4 M, about 5 M, about 6 M, about 7 M, about 8 M, about 9 M, about 10 M, about 15 M, about 20 M, about 25 M, about 30 M, about 35 M, about 40 M, about 45 M, about 50 M, about 75 M, about 100 M, or any range in between any two of the aforementioned concentrations, including said two concentrations as endpoints of the range, or any number in between any two of the aforementioned concentrations.

When administered orally, the active agent composition may be protected from digestion. This can be accomplished either by complexing the active agent with a composition to render it resistant to acidic and enzymatic hydrolysis or by packaging the active agent in an appropriately resistant carrier such as (but not limited to) a liposome, e.g., such as shown in U.S. Pat. No. 5,391,377.

In certain embodiments, the different therapeutic compounds of the disclosure can be administered within one hour of each other, within two hours of each other, within 3 hours of each other, within 6 hours of each other, within 12 hours of each other, within 24 hours of each other, within 36 hours of each other, within 48 hours of each other, within 72 hours of each other, or more. Thus an individual who receives such treatment can benefit from a combined effect of the different therapeutic compounds.

The active agents of the present disclosure can be administered to a subject by any of a number of effective routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compounds may also be formulated for inhalation. In certain embodiments, a compound may be simply dissolved or suspended in sterile water. Oral formulations may be formulated such that the active agents passes through a portion of the digestive system before being released, for example it may not be released until reaching the small intestine, or the colon. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

Tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations which are suitable for vaginal administration also include pessaries, tampons, creams, gels, pastes, foams or spray formulations containing such carriers as are known in the art to be appropriate. In certain embodiments, the active agents of the present disclosure can be formulated into suppositories, slow release formulations, or intrauterine delivery devices (IUDs).

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required. The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane. Transdermal patches have the added advantage of providing controlled delivery of a compound of the present disclosure to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this disclosure. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatable with such fluids. A particular (but non-limiting) route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. For topical transdermal administration, the agents are formulated into ointments, creams, salves, powders, and gels. Transdermal delivery systems can also include (for example but not by way of limitation) patches. The present compositions can also be administered in sustained delivery or sustained release mechanisms. For example, biodegradeable microspheres or capsules or other biodegradeable polymer configurations capable of sustained delivery can be included herein.

The compositions of the present disclosure may be formulated as implants, in the form of either biodegradable microparticles or small squared films comprising the microparticles of the present disclosure. The following describes methods of making such implants. Microparticles (e.g., 5-100 micrometers) containing different loadings of the compounds of the present disclosure, such as heteroarotinoids, are prepared by spray drying suspensions of the nanocrystals of the compounds and a biodegradable polymer (for example, polylactic acid of molecular weights 50,000-100,000) or polylactic-co-glycolic acid copolymer (e.g., proportions 75:25 or 50:50). Microparticles may contain, e.g., 10-50% wt/wt drug:polymer and can be implanted alone, or in a biodegradable film, e.g., as an implantable chitosan-egg phosphatidylcholine (ePC) films. To make such chitosan-egg phosphatidylcholine (ePC) films, chitosan flakes and ePC can be dissolved in a 1% acetic acid at a ratio of 1:0.8 (wt/wt). Microparticles containing the drug in nanocrystal form are dispersed in the chitosan-ePC solution in different proportions (e.g., 1:3, 1:5, 1:7 and 1:10 wt/wt) to achieve the release of different drug doses. The resulting microparticle-chitosan-ePC suspension can be poured into a Teflon dish to have a 2-3 mm thickness and allowed to dry in a covered dessicator for 5 days. After the films are dry, they can be cut into small squares of 15×15 mm². The implants can be made in different forms, including but not limited to thin films, rods, and wafers. Other biodegradable polymers that can be used to make implants include, but are not limited to, poly-lactic acid, poly-lactic-co-glycolic acid copolymer, poly-caprolactone, poly-sebacic acid, poly-adipic acid, poly (3-hydroxybutyric acid), poly(3-hydroxybutyrate-co-3-hydroxyvalerate, poly-trimethylene carbonate, chitosan, chitin, gelatin, collagen, and hyaluronic acid.

In non-limiting embodiments, gels comprising the active agents of the present disclosure can be made by combining the active agents in various proportions to a sodium alginate gel base or carbomer jelly base to form a homogeneous gel suitable for topical application. In non-limiting embodiments, ointments comprising the active agents of the present disclosure can be made by combining the active agents in various proportions to a Hydrophilic Petrolatum USP base, Lanolin, USP base or to Polyethylene glycol ointment, NF to form a homogeneous ointment suitable for topical application. In non-limiting embodiments, creams comprising the active agents of the present disclosure can be made by combining the active agents in various proportions in suspension in water and glycerin (e.g., 20:1 parts) and emulsified in a mixture of e.g., Lanolin, Beeswax USP-NF and Cetyl alcohol, plus a Tween 80 and Span 80. Several gel, ointment, and/or cream compositions that can be used are shown in Garcia-Contreras L, Abu-Izza K, Lu D R. “Biodegradable cisplatin microspheres for direct brain injection: Preparation and characterization.” Pharm Dev Technol (1997) 2(1): 53-65.

For inhalation, the present compositions can be delivered using any system known in the art, including (but not limited to) dry powder aerosols, liquids delivery systems, air jet nebulizers, propellant systems, and the like. For example (but not by way of limitation), the pharmaceutical formulation can be administered in the form of an aerosol or mist. For aerosol administration, the formulation can be supplied in finely divided form along with a surfactant and propellant. In another non-limiting aspect, the device for delivering the formulation to respiratory tissue is an inhaler in which the formulation vaporizes. Other liquid delivery systems include (for example but not by way of limitation) air jet nebulizers.

For inhalation, the present compositions can be delivered using any system known in the art, including (but not limited to) dry powder aerosols, liquids delivery systems, air jet nebulizers, propellant systems, and the like. For example (but not by way of limitation), the pharmaceutical formulation can be administered in the form of an aerosol or mist. For aerosol administration, the formulation can be supplied in finely divided form along with a surfactant and propellant. In another non-limiting aspect, the device for delivering the formulation to respiratory tissue is an inhaler in which the formulation vaporizes. Other liquid delivery systems include (for example, but not by way of limitation) air jet nebulizers.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

As noted, effective amounts of the active agents may be administered orally, in the form of a solid or liquid preparations such as capsules, pills, tablets, lozenges, melts, powders, suspensions, solutions, elixirs or emulsions. Solid unit dosage forms can be capsules of the ordinary gelatin type containing, for example, surfactants, lubricants, and inert fillers such as lactose, sucrose, and cornstarch, or the dosage forms can be sustained release preparations. The pharmaceutical composition may contain a solid carrier, such as a gelatin or an adjuvant. The tablet, capsule, and powder may contain from about 0.05 to about 95% of the active substance compound by dry weight. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol, or polyethylene glycol. When administered in liquid form, the pharmaceutical composition particularly contains from about 0.005 to about 95% by weight of the active agent(s). For example, a dose of about 10 mg to about 1000 mg once or twice a day could be administered orally.

In another embodiment, the active agents of the present disclosure can be tableted with conventional tablet bases such as lactose, sucrose, and cornstarch in combination with binders, such as acacia, cornstarch, or gelatin, disintegrating agents such as potato starch or alginic acid, and a lubricant such as stearic acid or magnesium stearate. Liquid preparations are prepared by dissolving the active agents in an aqueous or non-aqueous pharmaceutically acceptable solvent which may also contain suspending agents, sweetening agents, flavoring agents, and preservative agents as are known in the art.

In one non-limiting aspect, the active agent is incorporated in lipid monolayers or bilayers, such as (but not limited to) liposomes. Liposomes and liposomal formulations can be prepared according to standard methods and are also well known in the art, such as (but not limited to) those disclosed in U.S. Pat. Nos. 6,110,490; 6,096,716; 5,283,185; 5,279,833; 4,235,871; 4,501,728; and 4,837,028.

In one non-limiting aspect, the compositions are prepared with carriers that will protect the active agent against rapid elimination from the body, such as (but not limited to) a controlled release formulation, including implants and microencapsulated delivery systems. Biodegradable, biocompatible polymers can be used, such as (but not limited to) ethylene vinyl acetate, polyanhydrides, polyglycolic acid, collagen, polyorthoesters, and polylactic acid. Methods for preparation of such formulations will be apparent to those skilled in the art.

The active agents in general may be formulated to obtain compositions that include one or more pharmaceutically suitable excipients, surfactants, polyols, buffers, salts, amino acids, or additional ingredients, or some combination of these. This can be accomplished by known methods to prepare pharmaceutically useful dosages, whereby the active agent is combined in a mixture with one or more pharmaceutically suitable excipients. Sterile phosphate-buffered saline is one non-limiting example of a pharmaceutically suitable excipient.

The acid addition salts may include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, cam phorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N?-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, undecanoate, undecylenate, and valerate.

Non-limiting examples of routes of administration of the compositions described herein include parenteral injection, e.g., by subcutaneous, intramuscular, or transdermal delivery. Other forms of injection include (but are not limited to) intravenous, intraarterial, intralymphatic, intrathecal, intraocular, intranasal, intracranial, intracerebral, intraperitoneal, or intracavitary injection. In parenteral administration, the compositions will be formulated in a unit dosage injectable form such as (but not limited to) a solution, suspension, or emulsion, in association with a pharmaceutically acceptable excipient. Such excipients are inherently nontoxic and nontherapeutic. Non-limiting examples of such excipients include saline, Ringer's solution, dextrose solution, and Hanks' solution. Nonaqueous excipients such as (but not limited to) fixed oils and ethyl oleate may also be used. An alternative non-limiting excipient is 5% dextrose in saline. The excipient may contain minor amounts of additives such as (but not limited to) substances that enhance isotonicity and chemical stability, including buffers and preservatives. The active agents can be delivered or administered alone or as pharmaceutical compositions by any means known in the art, such as (but not limited to) systemically, regionally, or locally, for example by intraarterial, intrathecal (IT), intravenous (IV), parenteral, intrapleural cavity, topical, oral, or local administration, as subcutaneous, intratracheal (e.g., by aerosol) or transmucosal administration (e.g., buccal, bladder, vaginal, uterine, rectal, and/or nasal mucosa). Administration can also be localized directly into a tumor. Administration into the systemic circulation by intravenous, inhalation, mucosal, or subcutaneous administration is typical. Intravenous administration can be, for example (but not by way of limitation), by infusion over a period such as (but not limited to) 30-90 min or by a single bolus injection, or by other regimens as described elsewhere herein.

For parenteral administration, for example, the active agents may be dissolved in a physiologically acceptable pharmaceutical carrier and administered as either a solution or a suspension. Illustrative of suitable pharmaceutical carriers are water, saline, dextrose solutions, fructose solutions, ethanol, or oils of animal, vegetative, or synthetic origin. The pharmaceutical carrier may also contain preservatives and buffers as are known in the art.

When an effective amount of the active agents is administered by intravenous, cutaneous, or subcutaneous injection, the compound is particularly in the form of a pyrogen-free, parenterally acceptable aqueous solution or suspension. The preparation of such parenterally acceptable solutions, having due regard to pH, isotonicity, stability, and the like, is well within the skill in the art. A particular pharmaceutical composition for intravenous, cutaneous, or subcutaneous injection may contain, in addition to the active agent, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical compositions of the present disclosure may also contain stabilizers, preservatives, buffers, antioxidants, or other additives known to those of skill in the art.

As noted, particular amounts and modes of administration can be determined by one skilled in the art. One skilled in the art of preparing formulations can readily select the proper form and mode of administration, depending upon the particular characteristics of the active agents selected, the condition to be treated, the stage of the condition, and other relevant circumstances using formulation technology known in the art, described, for example, in Remington: The Science and Practice of Pharmacy, 22nd ed.

Additional pharmaceutical methods may be employed to control the duration of action of the active agents. Increased half-life and/or controlled release preparations may be achieved through the use of proteins or polymers to conjugate, complex with, and/or absorb the active agents as discussed previously herein. The controlled delivery and/or increased half-life may be achieved by selecting appropriate macromolecules (for example but not by way of limitation, polysaccharides, polyesters, polyamino acids, homopolymers, polyvinyl pyrrolidone, ethylenevinylacetate, methylcellulose, or carboxymethylcellulose, and acrylamides such as N-(2-hydroxypropyl) methacrylamide), and the appropriate concentration of macromolecules as well as the methods of incorporation, in order to control release.

Another possible method useful in controlling the duration of action of the active agents by controlled release preparations and half-life is incorporation of the active agents or their functional derivatives into particles of a polymeric material such as polyesters, polyamides, polyamino acids, hydrogels, poly(lactic acid), ethylene vinylacetate copolymers, copolymer micelles of, for example, polyethylene glycol (PEG) and poly(1-aspartamide). Additional pharmaceutical methods may be employed to increase bioavailability of the drug, such as Kolliphor HS15.

It is also possible to entrap the active agents in microcapsules prepared, for example, by coacervation techniques or by interfacial polymerization (for example, hydroxymethylcellulose or gelatine-microcapsules and poly-(methylmethacylate) microcapsules, respectively), in colloidal drug delivery systems (for example, liposomes, albumin microspheres, microemulsions, nano-particles, and nanocapsules), or in macroemulsions. Such techniques are well known to persons having ordinary skill in the art.

When the active agents are to be used as an injectable material, they can be formulated into a conventional injectable carrier. Suitable carriers include biocompatible and pharmaceutically acceptable phosphate buffered saline solutions, which are particularly isotonic.

For reconstitution of a lyophilized product in accordance with the present disclosure, one may employ a sterile diluent, which may contain materials generally recognized for approximating physiological conditions and/or as required by governmental regulation. In this respect, the sterile diluent may contain a buffering agent to obtain a physiologically acceptable pH, such as sodium chloride, saline, phosphate-buffered saline, and/or other substances which are physiologically acceptable and/or safe for use. In general, the material for intravenous injection in humans should conform to regulations established by the Food and Drug Administration, which are available to those in the field. The pharmaceutical composition may also be in the form of an aqueous solution containing many of the same substances as described above for the reconstitution of a lyophilized product.

The active agents can also be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, tauric acid, maleic acid, and fumaric acid, or by reaction with an inorganic base such as sodium hydroxide, ammonium hydroxide, potassium hydroxide, and organic bases such as monoalkyl, dialkyl, trialkyl and aryl amines, and substituted ethanolamines.

In certain embodiments, the present disclosure includes an active agent composition wherein at least one of the active agents is coupled (e.g., by covalent bond) directly or indirectly to a carrier molecule.

Formulated compositions comprising the active agents of the present disclosure can be provided in unit dosage form, e.g., in ampoules or in multi-dose containers, with an added preservative. Compositions can also take such forms as suspensions, solutions, or emulsions in oily or aqueous vehicles, and can contain formulatory agents such as suspending, stabilizing, and/or dispersing agents.

In some non-limiting methods, the patient is administered the active agent every one, two, three, or four weeks, for example. The dosage depends on the frequency of administration, condition of the patient, response to prior treatment (if any), whether the treatment is prophylactic or therapeutic, and whether the disorder is acute or chronic, among other factors.

The number of dosages administered may depends on the severity and temporal nature of the disorder (e.g., whether presenting acute or chronic symptoms) and the response of the disorder to the treatment. For acute disorders or acute exacerbations of a chronic disorder, between 1 and 10 doses may be used. Sometimes a single bolus dose, optionally in divided form, is sufficient for an acute disorder or acute exacerbation of a chronic disorder. Treatment can be repeated for recurrence of an acute disorder or acute exacerbation. For chronic disorders, the active agent may be administered at regular intervals, such as (but not limited to) weekly, fortnightly, monthly, quarterly, every six months for at least 1, 5, or 10 years, or for the life of the patient.

Compositions can be administered in a single dose treatment or in multiple dose treatments on a schedule and over a time period appropriate to the age, weight, and condition of the subject, the particular composition used, and the route of administration. In one non-limiting embodiment, a single dose of the composition according to the disclosure is administered. In other non-limiting embodiments, multiple doses are administered. The frequency of administration can vary depending on any of a variety of factors, e.g., severity of the symptoms, degree of immunoprotection desired, or whether the composition is used for prophylactic or curative purposes. For example, in certain non-limiting embodiments, the composition is administered once per month, twice per month, three times per month, every other week, once per week, twice per week, three times per week, four times per week, five times per week, six times per week, every other day, daily, twice a day, or three times a day. The duration of treatment (i.e., the period of time over which the composition is administered) can vary, depending on any of a variety of factors, e.g., subject response. For example (but not by way of limitation), the composition can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

The dosage of an administered active agent for humans will vary depending upon factors such as (but not limited to) the patient's age, weight, height, sex, general medical condition, and previous medical history. A dosage may be provided as several smaller amounts. For example, a single dosage of 500 mg may be administered as ten 50 mg tablets or capsules, or as five 100 mg tablets or capsules. The amounts of doses or dosages described herein may be provided in a single capsule, tablet, injection, infusion, or other more of delivery. Or, the amounts of drug which comprise the doses or dosages described herein may be provided in two or more (e.g., 2, 3, 4, 5, 6, 7, 8, 9, 10, 11, or 12) capsules, tablets, injections, infusions, or other modes of delivery.

In certain non-limiting embodiments, the recipient may be provided with a dosage of the active agent that is in the range of from about 1 mg to about 1000 mg. A lower or higher dosage also may be administered. In certain non-limiting embodiments, the dosage may be in the range of from about 25 mg to about 100 mg of the active agent per square meter (m²) of body surface area for a typical adult, although a lower or higher dosage also may be administered. Non-limiting examples of dosages of the active agent that may be administered to a human subject further include 1 to 750 mg, 1 to 600 mg, 1 to 500 mg, 1 to 400 mg, 1 to 300 mg, 1 to 200 mg, or 1 to 100 mg, although higher or lower doses may be used. Dosages may be repeated as needed, for example (but not by way of limitation), once per week for 4-10 weeks, once per week for 8 weeks, or once per week for 4 weeks. In certain non-limiting embodiments, the dosage can be provided as an infusion, for example as a single injection or as multiple injections. It may also be given less frequently, such as (but not limited to) every other week for several months, or more frequently, such as twice weekly, or by continuous infusion.

In some non-limiting embodiments, a per capsule or tablet dose may range from 25 mg to 200 mg (e.g., from 25 mg to 175 mg, from 25 mg to 150 mg, from 25 mg to 125 mg, from 25 mg to 100 mg, from 25 mg to 75 mg, from 25 mg to 70 mg, from 40 mg to 100 mg, from 40 mg to 75 mg, from 40 mg to 175 mg, from 40 mg to 150 mg, from 40 mg to 125 mg, from 40 mg to 70 mg, from 40 mg to 60 mg, or from 45 mg to 55 mg). In some non-limiting embodiments, the dose per capsule or tablet may be about 50 mg.

In some non-limiting embodiments, the dose per capsule or tablet is from 20 mg to 200 mg (e.g., from 20 mg to 175 mg, from 20 mg to 150 mg, from 20 mg to 100 mg, from 20 mg to 75 mg, from 30 mg to 175 mg, from 40 mg to 175 mg, from 45 mg to 175 mg, from 50 mg to 175 mg, from 30 mg to 150 mg, from 40 mg to 100 mg, from 45 mg to 75 mg, from 50 mg to 70 mg, from 55 mg to 65 mg, from 58 mg to 62 mg; about 20 mg, about 30 mg, about 45 mg, or e.g., about 60 mg). In some non-limiting embodiments, the dose is from about 12 mg to about 48 mg (e.g., from about 12 mg to about 42 mg, from about 12 mg to about 36 mg, from about 12 mg to about 30 mg, from about 18 mg to about 48 mg, from about 18 mg to about 42 mg, from about 24 mg to about 42 mg, from about 27 mg to about 42 mg, from about 24 mg to about 36 mg, from about 27 mg to about 33 mg, from about 28 mg to about 32 mg; e.g., about 24 mg, about 27 mg, about 30 mg, about 33 mg, or about 36 mg).

In some non-limiting embodiments, the dosing regimen comprises administration of a loading dose, such as from 20 mg to 200 mg (e.g., from 20 mg to 175 mg, from 20 mg to 150 mg, from 20 mg to 100 mg, from 20 mg to 75 mg, from 30 mg to 175 mg, from 40 mg to 175 mg, from 45 mg to 175 mg, from 50 mg to 175 mg, from 30 mg to 150 mg, from 40 mg to 100 mg, from 45 mg to 75 mg, from 50 mg to 70 mg, from 55 mg to 65 mg, from 58 mg to 62 mg; e.g., about 60 mg). In some non-limiting embodiments, the dose is from about 12 mg to about 48 mg (e.g., from about 12 mg to about 42 mg, from about 12 mg to about 36 mg, from about 12 mg to about 30 mg, from about 18 mg to about 48 mg, from about 18 mg to about 42 mg, from about 24 mg to about 42 mg, from about 27 mg to about 42 mg, from about 24 mg to about 36 mg, from about 27 mg to about 33 mg, from about 28 mg to about 32 mg; e.g., about 24 mg, about 27 mg, about 30 mg, about 33 mg, or about 36 mg).

In certain non-limiting embodiments, the per day dosage for administration to a subject is in a range of about 1 mg/kg to about 25 mg/kg, or about 2 mg/kg to about 24 mg/kg, or about 3 mg/kg to about 22 mg/kg, or about 4 mg/kg to about 20 mg/kg, or about 5 mg/kg to about 17.5 mg/kg, or about 5 mg/kg to about 15 mg/kg, or about 5 mg/kg to about 12.5 mg/kg, or about 6 mg/kg to about 25 mg/kg, or about 6 mg/kg to about 20 mg/kg, or about 6 mg/kg to about 15 mg/kg, or about 6 mg/kg to about 12 mg/kg.

In certain non-limiting embodiments, the amount of the active agent delivered to the subject per dose is in a range of about 100 mg to about 1000 mg, or about 200 mg to about 1000 mg, or about 300 mg to about 1000 mg, or about 400 mg to about 1000 mg, or about 250 mg to about 900 mg, or about 400 mg to about 900 mg, or about 400 mg to about 850 mg, or about 400 mg to about 800 mg, or about 400 mg to about 700 mg, or about 420 mg to about 840 mg, or about 500 mg to about 650 mg, or about 500 mg to about 1000 mg, or about 500 mg to about 900 mg, or about 500 mg to about 850 mg, or about 500 mg to about 800 mg, or about 500 mg to about 700 mg. The total dose to be delivered can be provided in a single capsule, tablet, injection, or bolus (or other dosage form), or in multiple capsules, tablets, injections, or boluses (or other dosage forms).

In at least certain non-limiting embodiments, when the active agent is provided in the form of a capsule or tablet, the capsule or tablet should disintegrate within about 5-10 minutes, and in the gastrointestinal (GI) tract the active agent should dissolve in about 30 minutes. In at least certain non-limiting embodiments, once the active agents are absorbed from the GI tract, they should produce therapeutic concentrations in the target tissue in the subject for at least about 4, to about 8, to about 12, to about 16, to about 20, or to about 24 hours, when the active agent is administered once a day.

In certain non-limiting embodiments of the cancer treatment methods, drug combinations, and kits and packages of the present disclosure, the following heteroarotinoid drug+MYC inhibitor drug combinations may be used: SHetA2+MYCi975, SHetA2+OMO-103, SHetA2+WBC100, SHetA2+MYCi361, SHetA2+10058-F4, SHetA2+10074-G5, SHetA2+MYCMI-6, SHetA2+BTYNB, SHetA2+EN4, SHetA2+APTO-253, SHetA2+CX5461, SHetA2+ML327, SHetA2+Mycro 3, SHetA2+KJ Pyr 9, SHetA2+SaJM589, SHetA2+KSI-3716, SHetA2+KI-MS2-008, SHetA2+Triptolide, SHetA2+MLN8237 (alisertib), SHetA2+CD532, SHetA2+BI-6727 (a.k.a. Volasertib), SHetA2+JQ1, SHetA2+OTX015/MK-8628, SHetA2+GSK2820151, SHetA2+ZEN-3694, SHetA2+CC-90010, SHetA2+ABBV-744, SHetA2+ABBV-075 (mivebresib), SHetA2+BI-894999, SHetA2+CPI-0610 (Pelabresib), SHetA2+GSK525762/I-BET762, SHetA2+INCB057643, SHetA2+dBET6, SHetA2+Alobresib, SHetA2+Inobrodib, SHetA2+BI-2536, SHetA2+LB-100, SHetA2+SMAP(DT-061), SHetA2+FTY720, SHetA2+OP449, SHetA2+perphenazine, SHetA2+KPT-6566, SHetA2+ATRA, SHetA2+BJP-06-005-3, SHetA2+Sulfopin, SHetA2+AZD1208, SHetA2+TP-3654, SHetA2+PIM447, SHetA2+SGI-1776, SHetA2+LGH447, SHetA2+SEL24/MEN1703, SHetA2+Everolimus, SHetA2+Paxalisib, SHetA2+ATG-008, SHetA2+Gedatolisib, SHetA2+Samotolisib, SHetA2+Sapanisertib, SHetA2+Apitolisib, SHetA2+Zotatifin, SHetA2+TAK-981, and SHetA2+MRT-2359.

Various embodiments of the present disclosure will be more readily understood by reference to the following examples and description, which as noted above are included merely for purposes of illustration of certain aspects and embodiments of the present disclosure, and are not intended to be limiting. The following detailed examples and methods which describe various compositions of the present disclosure and are to be construed, as noted above, only as illustrative, and not limitations of the disclosure in any way whatsoever.

### Methods and Results

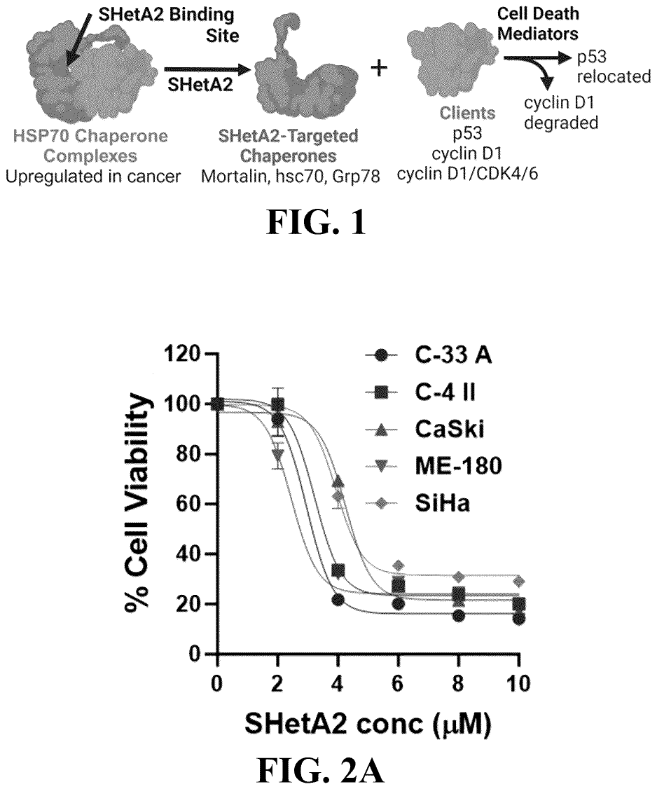

SHetA2 kills cancer cells without harming healthy cells by binding to three heat shock protein A (HSPA; 5/Grp78, 8/hsc70, and 9/mortalin). These are elevated in cancer cells while present at very low levels in healthy cells, thus representing cancer-selective SHetA2 targets and explaining the lack of SHetA2 toxicity in extensive preclinical studies. SHetA2 binding to these HSP70s causes the release of their client proteins, which then become susceptible to protein degradation and intracellular relocation (FIG. 1).

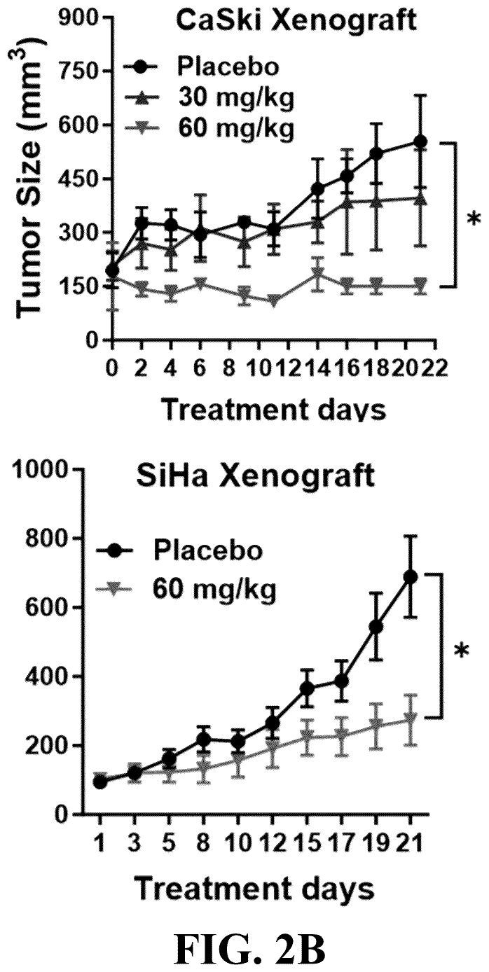

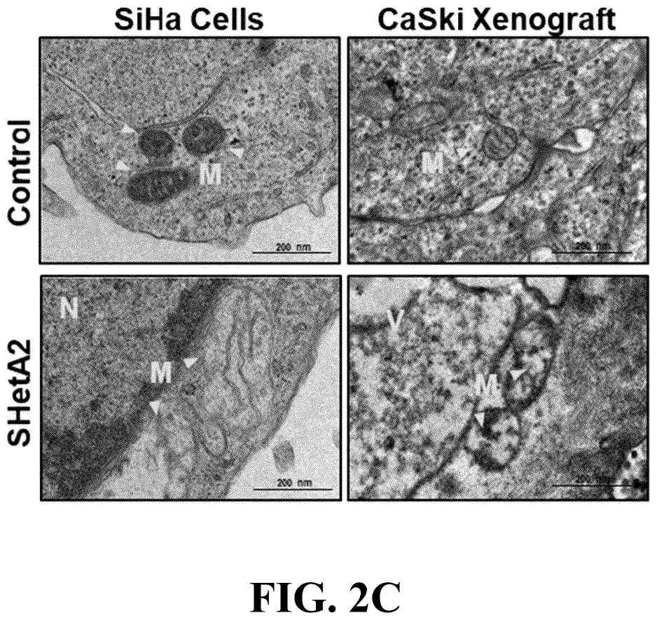

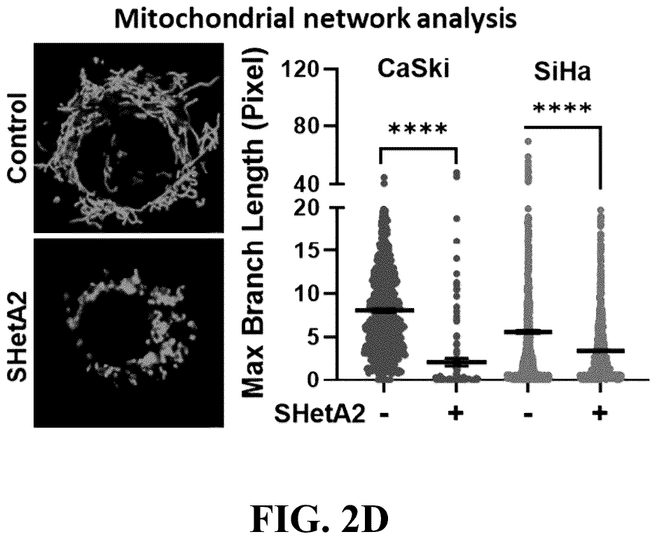

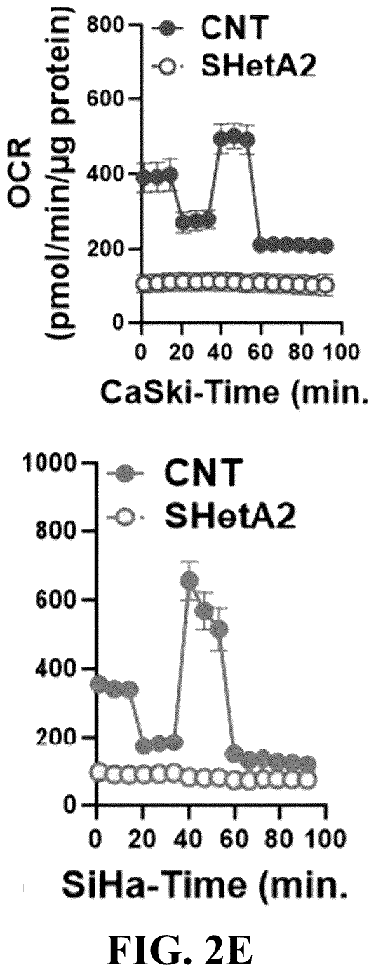

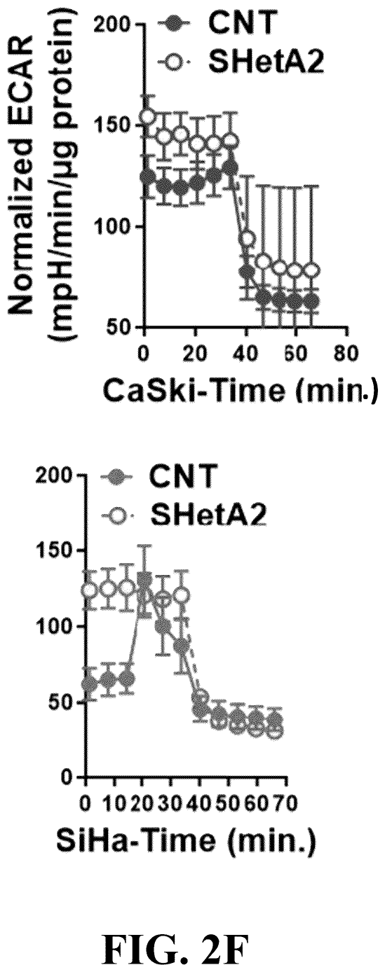

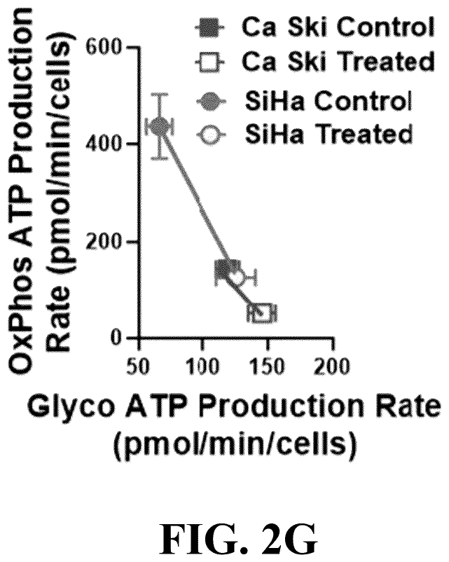

SHetA2 has been shown to have potent anticancer activity against cervical and ovarian cancers (FIGS. 2A-2B) and endometrial cancers without causing toxicity. While in endometrial and ovarian cancer, SHetA2 induced caspase-dependent apoptosis in cervical cancer SHetA2 caused extensive mitochondrial damage (FIGS. 2C-2D), leading to excessive mitophagy and DNA damage contributing to cell death.

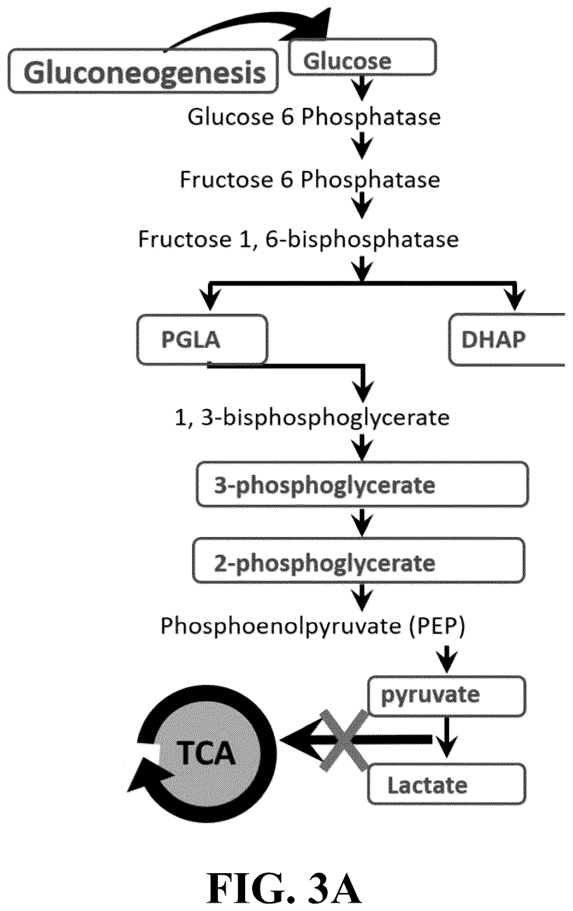

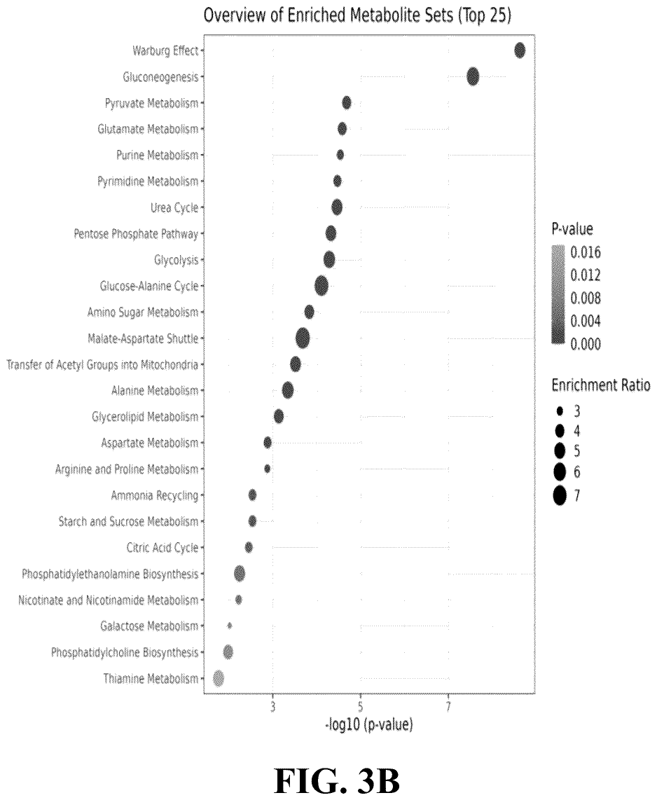

In cervical cancer cells, mitochondrial damage was associated with reduced oxidative phosphorylation (OxPhos, OCR, FIG. 2E) and upregulated glycolysis (increased extracellular acidification rate; ECAR, FOG. FIG. 2F). The energy map also demonstrated that SHetA2 decreased mitochondrial ATP production while increased glycolytic ATP production (FIG. 2G), demonstrating that cervical cancer cells respond to SHetA2 reduction of OxPhos by maximizing glycolysis. SHetA2-induced glycolysis in cervical cancer was further validated by liquid chromatography-coupled tandem mass spectrometry (LC-MS/MS) analysis demonstrating a significant increase in glycolytic intermediate metabolites (FIG. 3A, enclosed in boxes) and the Warburg effect (aerobic glycolysis for energy generation) as the top metabolic pathway altered by SHetA2 (FIG. 3B).

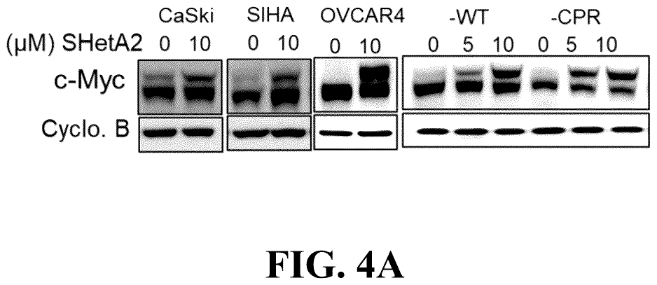

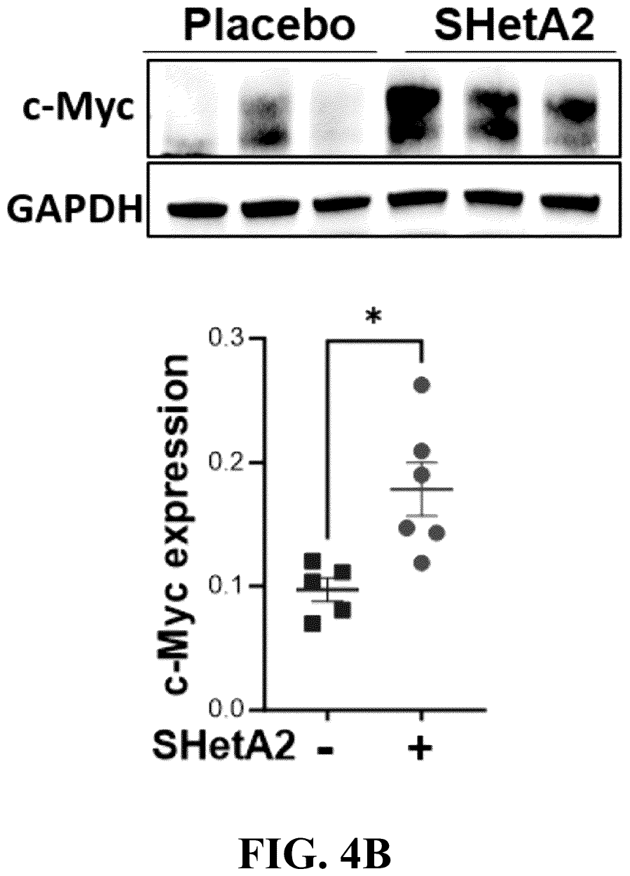

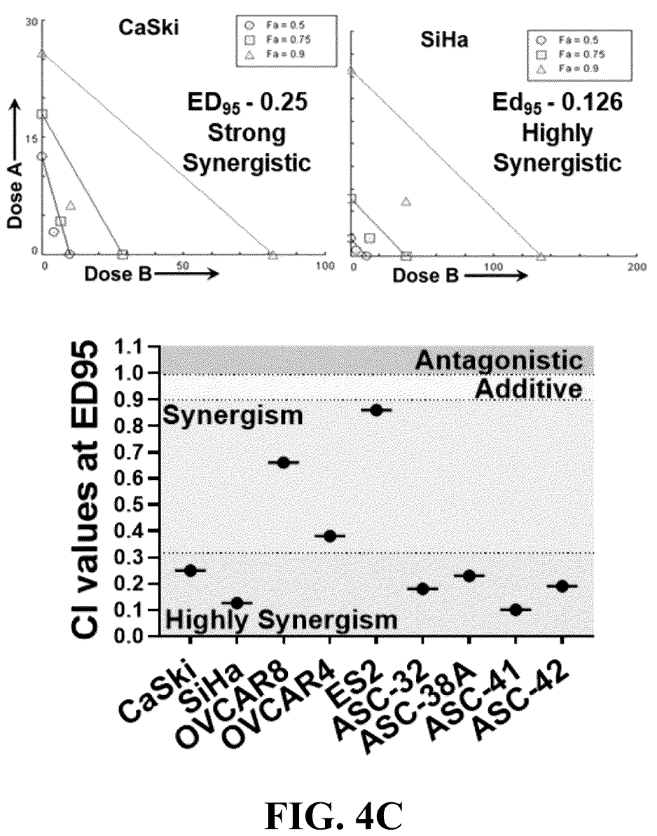

SHetA2 upregulated Myc expression in cervical and ovarian cancer cell lines (FIG. 4A) and cervical cancer xenograft models (FIG. 4B). Moreover, the combination of SHetA2 and a selective Myc inhibitor (Myci975, which inhibits Myc and MAX interaction) synergistically inhibited the viability of cancer cells (ovarian and cervical) and ascites spheroids (ASC) collected from ovarian cancer patients (FIG. 4C).

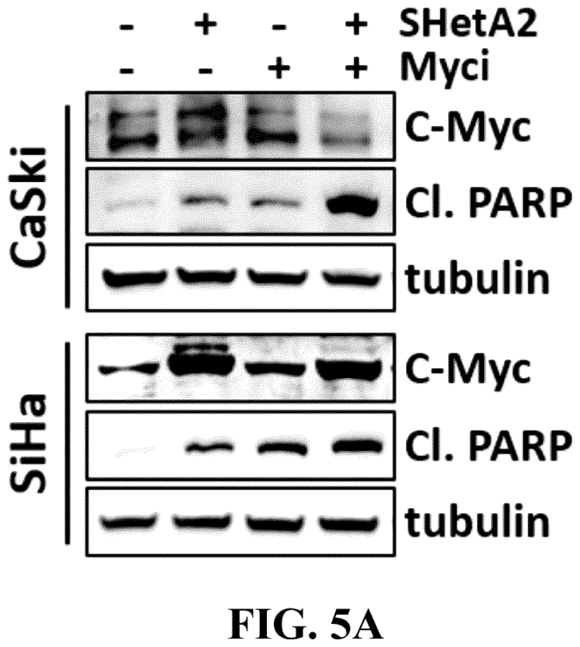

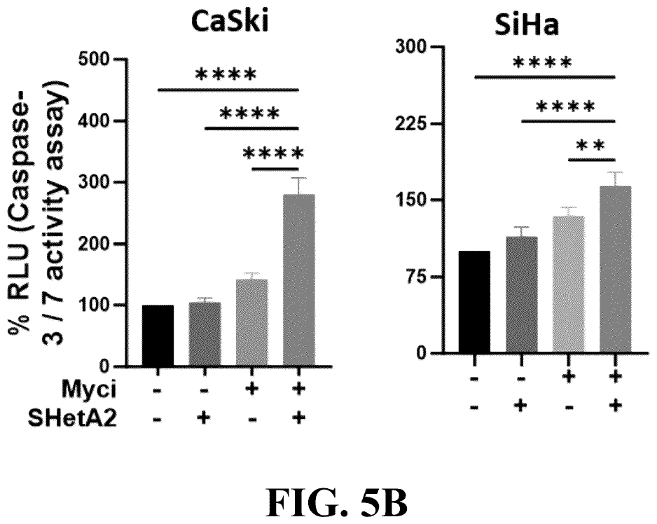

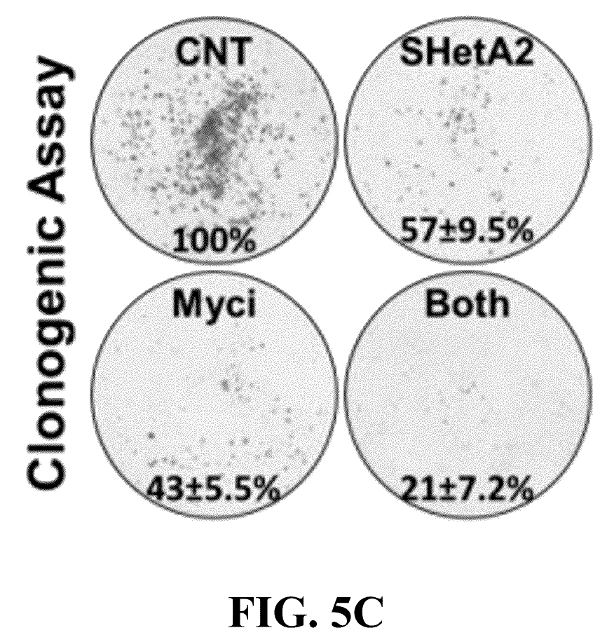

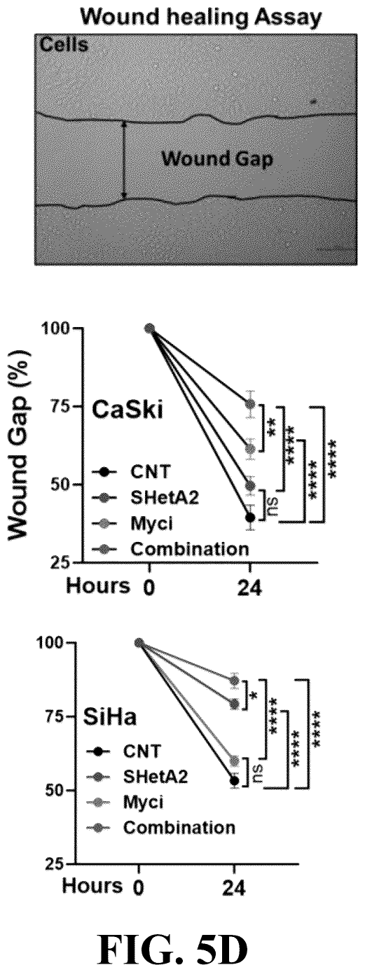

In cervical cancer cells, the combination of SHetA2 and Myci975 significantly increased the expression of cleaved (cl) PARP (FIG. 5A) and caspase-3 activity (FIG. 5B) in association with reduction in SHetA2-induced Myc expression. In addition, SHetA2 and Myci975 combination inhibited clonogenic activity (FIG. 5C) and migration ability (FIG. 5D, wound healing assay) as compared to individual drugs or control.

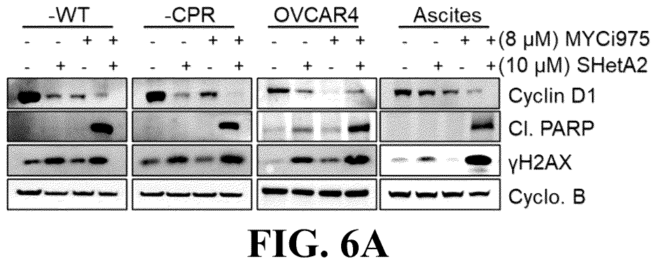

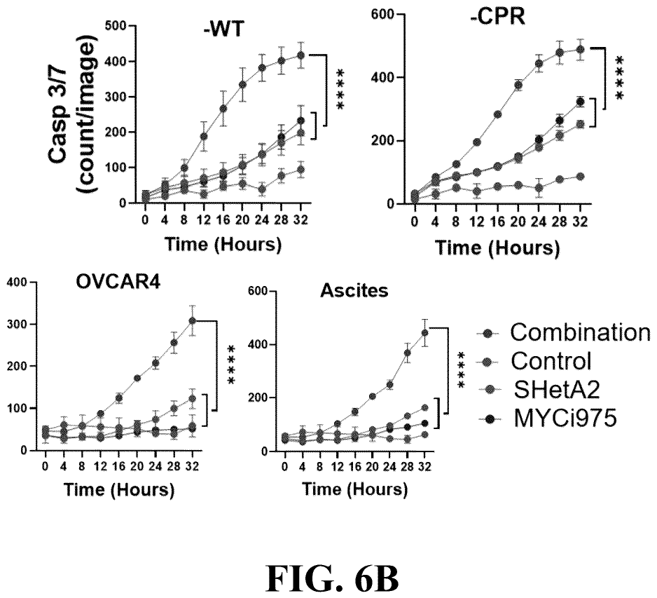

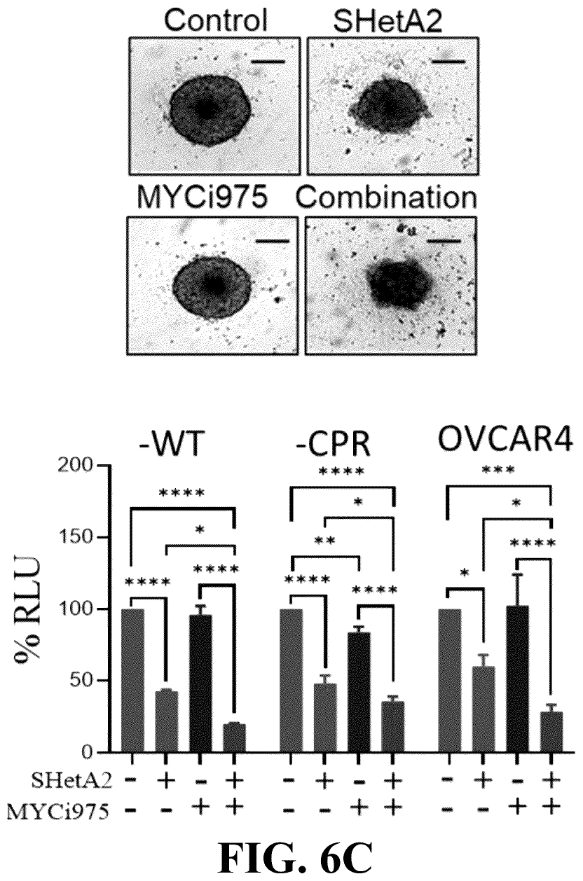

Moreover, SHetA2 in combination MYCi975 synergistically downregulated the expression of proliferative marker (cyclinD1) and induced apoptosis as evident by increased expression of cleaved PARP, YH2AX and caspase 3 activity in OvCa cells and ASC collected from OvCa patients (FIGS. 6A-6C).

These results indicate that the Myc inhibitor synergistically enhances the therapeutic efficacy of SHetA2 by counteracting the Myc-associated growth and metabolic pathway with low-to-no toxicity in cervical and ovarian cancers.

Using cervical (SiHa) and ovarian (OVCAR8-CPR) xenograft models, we evaluated the therapeutic efficacy of SHetA2 and MYCi975, alone and in combination.

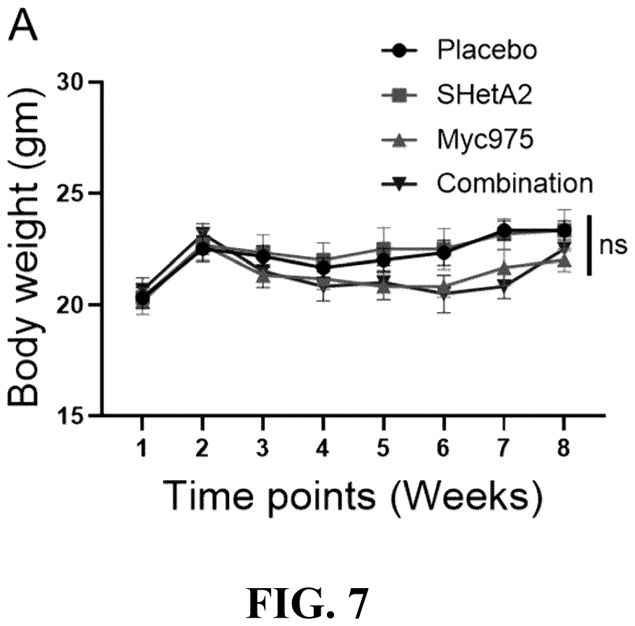

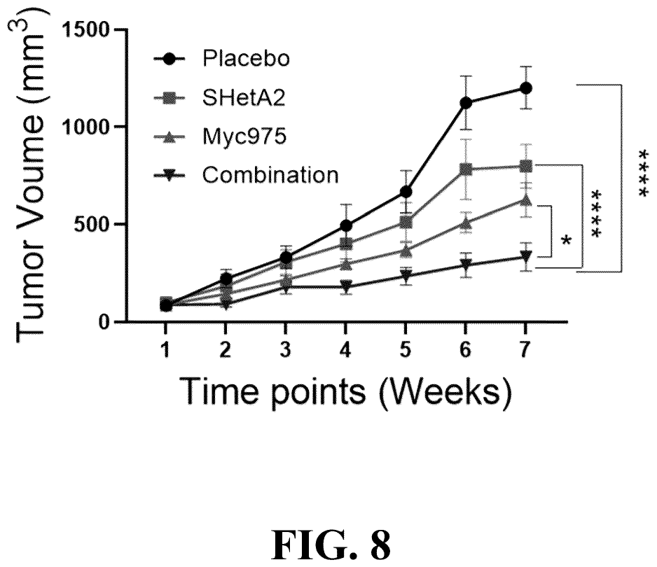

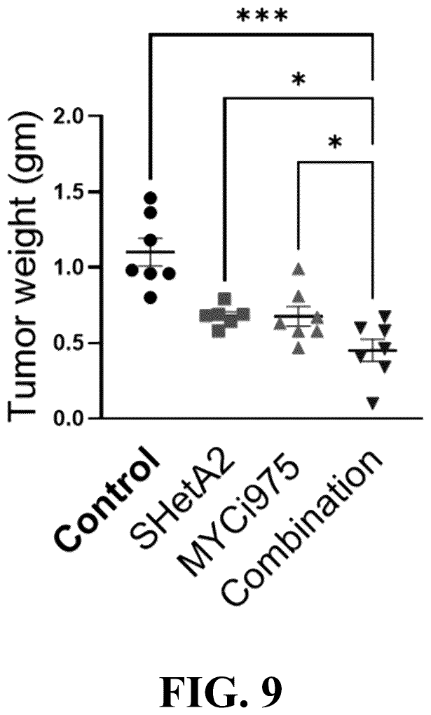

In the SiHa model, body weight remained stable across all treatment groups (FIG. 7), indicating that all regimens were well tolerated. Combination treatment produced greater tumor growth inhibition than either agent alone, as shown by reduced tumor volume over time (FIG. 8) and significantly lower endpoint tumor weights (FIG. 9).

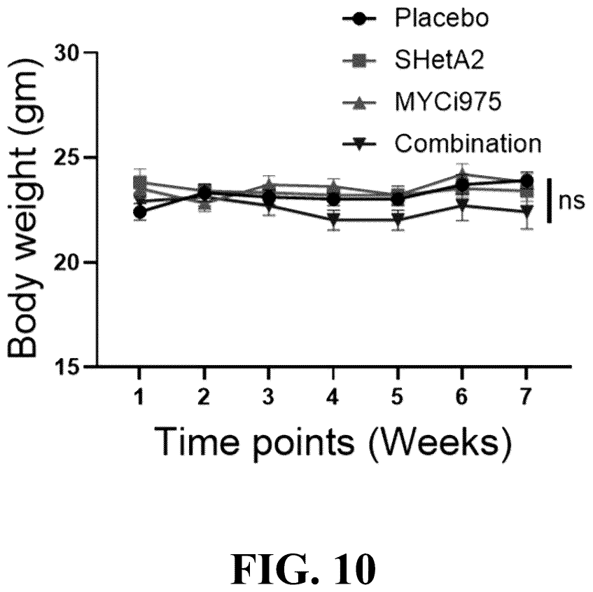

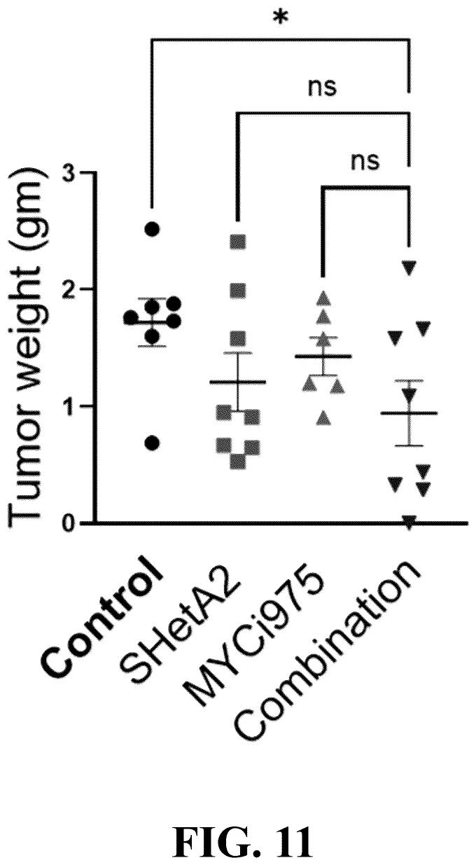

Similarly, in the OVCAR8-CPR ovarian cancer xenograft model, body weights remained stable across all groups, indicating minimal systemic toxicity (FIG. 10). While single-agent treatments had limited effects, combination therapy significantly reduced endpoint tumor weights (FIG. 11). Together, these results demonstrate that a combination of SHetA2 and MYCi975 synergistically inhibits tumor growth in vivo without detectable toxicity in cervical or ovarian cancer xenograft models.

While the present disclosure has been described in connection with certain embodiments so that aspects thereof may be more fully understood and appreciated, it is not intended that the present disclosure be limited to these particular embodiments. On the contrary, it is intended that all alternatives, modifications and equivalents are included within the scope of the present disclosure. Thus the examples described above, which include particular embodiments, will serve to illustrate the practice of the present disclosure, it being understood that the particulars shown are by way of example and for purposes of illustrative discussion of particular embodiments only and are presented in the cause of providing what is believed to be the most useful and readily understood description of procedures as well as of the principles and conceptual aspects of the presently disclosed methods and compositions. Changes may be made in the formulation of the various compositions described herein, the methods described herein or in the steps or the sequence of steps of the methods described herein without departing from the spirit and scope of the present disclosure.

### REFERENCES

The following references, to the extent that they provide exemplary procedural or other details supplementary to those set forth herein, are specifically incorporated herein by reference. In addition, the following is not intended to be an Information Disclosure Statement; rather, an Information Disclosure Statement in accordance with the provisions of 37 CFR § 1.97 will be submitted separately.
ADDIN EN.REFLIST 1. Benbrook, D. M. et al. SHetA2 interference with mortalin binding to p66shc and p53 identified using drug-conjugated magnetic microspheres. Invest New Drugs, 412-423. [Selected for inclusion into GLOBAL MEDICAL DISCOVERY [ISSN 1929-8536][https://globalmedicaldiscovery.com]. (2013).
2. Kabirov, K. K. et al. Oral toxicity and pharmacokinetic studies of SHetA2, a new chemopreventive agent, in rats and dogs. Drug and Chemical Toxicology 36, 284-295 (2012).
3. Benbrook, D. et al. Flexible heteroarotinoids (Flex-Hets) exhibit improved therapeutic ratios as anti-cancer agents over retinoic acid receptor agonists. Investigational New Drugs 23, 417-428 (2005).
4. Kennedy, A. L. et al. Complementary Targeting of Rb Phosphorylation and Growth in Cervical Cancer Cell Cultures and a Xenograft Mouse Model by SHetA2 and Palbociclib. Cancers (Basel) 12 (2020).
5. Chandra, V., Rai, R. & Benbrook, D. M. Utility and Mechanism of SHetA2 and Paclitaxel for Treatment of Endometrial Cancer. Cancers (Basel) 13 (2021).
6. Ramraj, S. K. et al. Novel ovarian cancer maintenance therapy targeted at mortalin and mutant p53. Int J Cancer 147, 1086-1097 (2020).
7. Rai, R., Lightfoot, S. & Benbrook, D. M. Manipulation of metabolic responses enhances SHetA2 efficacy without toxicity in cervical cancer cell lines and xenografts. Gynecol Oncol 180, 44-54 (2023).
8. Sitarz, K., Czamara, K., Szostek, S. & Kaczor, A. The impact of HPV infection on human glycogen and lipid metabolism—a review. Biochimica et Biophysica Acta (BBA)—Reviews on Cancer 1877, 188646 (2022).
9. Li, B. & Sui, L. Metabolic reprogramming in cervical cancer and metabolomics perspectives. Nutr Metab (Lond) 18, 93 (2021).
10. Peng, X. et al. Autophagy promotes paclitaxel resistance of cervical cancer cells: involvement of Warburg effect activated hypoxia-induced factor 1-α-mediated signaling. Cell Death Dis 5, e1367 (2014).

What is claimed is:

1. A cancer treatment method for treating a subject in need of such therapy, comprising:
conjointly administering a therapeutically-effective amount of at least one heteroarotinoid compound and at least one MYC inhibitor, wherein the therapeutically-effective amount yields a synergistic effect compared to an effect of the at least one heteroarotinoid alone and an effect of the at least one MYC inhibitor alone.

2. The cancer treatment method of claim 1, wherein the cancer treatment method results in at least one of reduction of tumor size, reduction of tumor growth, inhibition of cancer metastases, cancer cell death, and inhibition of tumor recurrence.

3. The cancer treatment method of claim 1, wherein the at least one heteroarotinoid compound is selected from the group consisting of SHetA2, SHetA3, SHetA4, SHetC2, SHetD3, SHetD4, SHetD5, SHet50, SHet65, SHet100, OHet72, NHet17, NHet86, NHet90, and combinations thereof.

4. The cancer treatment method of claim 1, wherein the at least one MYC inhibitor is selected from the group consisting of MYCi975, OMO-103, WBC-100, MYCi361, 10058-F4, 10074-G5, MYCMI-6, BTYNB, EN4, APTO-253, CX5461, ML327, Mycro 3, KJ Pyr 9, SaJM589, KSI-3716, KI-MS2-008, Triptolide, MLN8237, CD532, BI-6727, JQ1, OTX015, GSK2820151, ZEN-3694, CC-90010, ABBV-744, ABBV-075, BI-894999, CPI-0610, GSK525762, INCB057643, dBET6, Alobresib, Inobrodib, BI-2536, LB-100, SMAP, FTY720, OP449, perphenazine, KPT-6566, ATRA, BJP-06-005-3, Sulfopin, AZD1208, TP-3654, PIM447, SGI-1776, LGH447, SEL24, Everolimus, Paxalisib, ATG-008, Gedatolisib, Samotolisib, Sapanisertib, Apitolisib, Zotatifin, TAK-981, MRT-2359, and combinations thereof.

5. The cancer treatment method of claim 1, wherein the at least one heteroarotinoid compound and the at least one MYC inhibitor are selected from the group of combinations consisting of: SHetA2 and MYCi975, SHetA2 and OMO-103, SHetA2 and WBC100, SHetA2 and MYCi361, SHetA2 and 10058-F4, SHetA2 and 10074-G5, SHetA2 and MYCMI-6, SHetA2 and BTYNB, SHetA2 and EN4, SHetA2 and APTO-253, SHetA2 and CX5461, SHetA2 and ML327, SHetA2 and Mycro 3, SHetA2 and KJ Pyr 9, SHetA2 and SaJM589, SHetA2 and KSI-3716, SHetA2 and KI-MS2-008, SHetA2 and Triptolide, SHetA2 and MLN8237, SHetA2 and CD532, SHetA2 and BI-6727, SHetA2 and JQ1, SHetA2 and OTX015, SHetA2+GSK2820151, SHetA2 and ZEN-3694, SHetA2 and CC-90010, SHetA2 and ABBV-744, SHetA2 and ABBV-075, SHetA2 and BI-894999, SHetA2 and CPI-0610, SHetA2 and GSK525762, SHetA2 and INCB057643, SHetA2 and dBET6, SHetA2 and Alobresib, SHetA2 and Inobrodib, SHetA2 and BI-2536, SHetA2 and LB-100, SHetA2 and SMAP, SHetA2 and FTY720, SHetA2 and OP449, SHetA2 and perphenazine, SHetA2 and KPT-6566, SHetA2 and ATRA, SHetA2 and BJP-06-005-3, SHetA2 and Sulfopin, SHetA2 and AZD1208, SHetA2 and TP-3654, SHetA2 and PIM447, SHetA2 and SGI-1776, SHetA2 and LGH447, SHetA2 and SEL24, SHetA2 and Everolimus, SHetA2 and Paxalisib, SHetA2 and ATG-008, SHetA2 and Gedatolisib, SHetA2 and Samotolisib, SHetA2 and Sapanisertib, SHetA2 and Apitolisib, SHetA2 and Zotatifin, SHetA2 and TAK-981, SHetA2 and MRT-2359, and combinations thereof.

6. The cancer treatment method of claim 1, wherein the at least one MYC inhibitor compries a c-MYC inhibitor.

7. The cancer treatment method of claim 1, wherein the at least one heteroarotinoid compound comprises SHetA2, and the at least one MYC inhibitor comprises a c-MYC inhibitor.

8. The cancer treatment method of claim 1, wherein the at least one heteroarotinoid compound is administered in a dose range of about 1 mg/kg to about 100 mg/kg, and the at least one MYC inhibitor is administered in a dose range of about 1 mg/kg to about 100 mg/kg.

9. The cancer treatment method of claim 1, wherein the at least one heteroarotinoid compound and the at least one MYC inhibitor are administered in a weight-to-weight ratio in a range of about 50:1 to about 1:50.

10. A drug combination, comprising:
at least one heteroarotinoid compound; and
at least one MYC inhibitor;
wherein the at least one heteroarotinoid compound and the at least one MYC inhibitor are combined in a single composition, or are present in a kit of parts comprising at least two separate compositions for sequential or simultaneous administration to a subject.

11. The drug combination of claim 10, wherein the inhibition of cancer cells results in at least one of reduction of tumor size, reduction of tumor growth, inhibition of cancer metastases, inhibition of tumor recurrence, and cancer cell death.

12. The drug combination of claim 10, wherein the at least one heteroarotinoid compound is selected from the group consisting of SHetA2, SHetA3, SHetA4, SHetC2, SHetD3, SHetD4, SHetD5, SHet50, SHet65, SHet100, OHet72, NHet17, NHet86, NHet90, and combinations thereof.

13. The drug combination of claim 10, wherein the at least one MYC inhibitor is selected from the group consisting of MYCi975, OMO-103, WBC100, MYCi361, 10058-F4, 10074-G5, MYCMI-6, BTYNB, EN4, APTO-253, CX5461, ML327, Mycro 3, KJ Pyr 9, SaJM589, KSI-3716, KI-MS2-008, Triptolide, MLN8237, CD532, BI-6727, JQ1, OTX015, GSK2820151, ZEN-3694, CC-90010, ABBV-744, ABBV-075, BI-894999, CPI-0610, GSK525762, INCB057643, dBET6, Alobresib, Inobrodib, BI-2536, LB-100, SMAP, FTY720, OP449, perphenazine, KPT-6566, ATRA, BJP-06-005-3, Sulfopin, AZD1208, TP-3654, PIM447, SGI-1776, LGH447, SEL24, Everolimus, Paxalisib, ATG-008, Gedatolisib, Samotolisib, Sapanisertib, Apitolisib, Zotatifin, TAK-981, MRT-2359, and combinations thereof.

14. The drug combination of claim 10, wherein the at least one heteroarotinoid compound and the MYC inhibitor are selected from the group of combinations consisting of: SHetA2 and MYCi975, SHetA2 and OMO-103, SHetA2 and WBC100, SHetA2 and MYCi361, SHetA2 and 10058-F4, SHetA2 and 10074-G5, SHetA2 and MYCMI-6, SHetA2 and BTYNB, SHetA2 and EN4, SHetA2 and APTO-253, SHetA2 and CX5461, SHetA2 and ML327, SHetA2 and Mycro 3, SHetA2 and KJ Pyr 9, SHetA2 and SaJM589, SHetA2 and KSI-3716, SHetA2 and KI-MS2-008, SHetA2 and Triptolide, SHetA2 and MLN8237, SHetA2 and CD532, SHetA2 and BI-6727, SHetA2 and JQ1, SHetA2 and OTX015, SHetA2+GSK2820151, SHetA2 and ZEN-3694, SHetA2 and CC-90010, SHetA2 and ABBV-744, SHetA2 and ABBV-075, SHetA2 and BI-894999, SHetA2 and CPI-0610, SHetA2 and GSK525762, SHetA2 and INCB057643, SHetA2 and dBET6, SHetA2 and Alobresib, SHetA2 and Inobrodib, SHetA2 and BI-2536, SHetA2 and LB-100, SHetA2 and SMAP, SHetA2 and FTY720, SHetA2 and OP449, SHetA2 and perphenazine, SHetA2 and KPT-6566, SHetA2 and ATRA, SHetA2 and BJP-06-005-3, SHetA2 and Sulfopin, SHetA2 and AZD1208, SHetA2 and TP-3654, SHetA2 and PIM447, SHetA2 and SGI-1776, SHetA2 and LGH447, SHetA2 and SEL24, SHetA2 and Everolimus, SHetA2 and Paxalisib, SHetA2 and ATG-008, SHetA2 and Gedatolisib, SHetA2 and Samotolisib, SHetA2 and Sapanisertib, SHetA2 and Apitolisib, SHetA2 and Zotatifin, SHetA2 and TAK-981, SHetA2 and MRT-2359, and combinations thereof.

15. The drug combination of claim 10, wherein the at least one MYC inhibitor comprises a c-MYC inhibitor.

16. The drug combination of claim 10, wherein the at least one heteroarotinoid compound comprises SHetA2, and the at least one MYC inhibitor comprises a c-MYC inhibitor.

17. The drug combination of claim 10, wherein the at least one heteroarotinoid compound comprises a dosage in a range of about 1 mg/kg to about 100 mg/kg, and the at least one MYC inhibitor comprises a dosage in a range of about 1 mg/kg to about 100 mg/kg.

18. The drug combination of claim 10, wherein the at least one heteroarotinoid compound and the at least one MYC inhibitor comprise a weight-to-weight ratio in a range of about 50:1 to about 1:50.

19. A kit or commercial package, comprising:
at least one heteroarotinoid compound; and
at least one MYC inhibitor;
wherein the at least one heteroarotinoid and the at least one MYC inhibitor when administered conjointly provide a synergistic effect against cancer cells compared to an effect of the at least one heteroarotinoid alone and an effect of the at least one MYC inhibitor alone.

20. The kit or commercial package of claim 19, wherein the at least one heteroarotinoid compound is selected from the group consisting of SHetA2, SHetA3, SHetA4, SHetC2, SHetD3, SHetD4, SHetD5, SHet50, SHet65, SHet100, OHet72, NHet17, NHet86, NHet90, and combinations thereof.

21. The kit or commercial package of claim 19, wherein the at least one MYC inhibitor is selected from the group consisting of OMO-103, MYCi975, WBC100, MYCi361, 10058-F4, 10074-G5, MYCMI-6, BTYNB, EN4, APTO-253, CX5461, ML327, Mycro 3, KJ Pyr 9, SaJM589, KSI-3716, KI-MS2-008, Triptolide, MLN8237, CD532, BI-6727, JQ1, OTX015, GSK2820151, ZEN-3694, CC-90010, ABBV-744, ABBV-075, BI-894999, CPI-0610, GSK525762, INCB057643, dBET6, Alobresib, Inobrodib, BI-2536, LB-100, SMAP, FTY720, OP449, perphenazine, KPT-6566, ATRA, BJP-06-005-3, Sulfopin, AZD1208, TP-3654, PIM447, SGI-1776, LGH447, SEL24, Everolimus, Paxalisib, ATG-008, Gedatolisib, Samotolisib, Sapanisertib, Apitolisib, Zotatifin, TAK-981, MRT-2359, and combinations thereof.

22. The kit or commercial package of claim 19, wherein the at least one heteroarotinoid compound and the at least one MYC inhibitor are selected from the group of combinations consisting of: SHetA2 and MYCi975, SHetA2 and OMO-103, SHetA2 and WBC100, SHetA2 and MYCi361, SHetA2 and 10058-F4, SHetA2 and 10074-G5, SHetA2 and MYCMI-6, SHetA2 and BTYNB, SHetA2 and EN4, SHetA2 and APTO-253, SHetA2 and CX5461, SHetA2 and ML327, SHetA2 and Mycro 3, SHetA2 and KJ Pyr 9, SHetA2 and SaJM589, SHetA2 and KSI-3716, SHetA2 and KI-MS2-008, SHetA2 and Triptolide, SHetA2 and MLN8237, SHetA2 and CD532, SHetA2 and BI-6727, SHetA2 and JQ1, SHetA2 and OTX015, SHetA2+GSK2820151, SHetA2 and ZEN-3694, SHetA2 and CC-90010, SHetA2 and ABBV-744, SHetA2 and ABBV-075, SHetA2 and BI-894999, SHetA2 and CPI-0610, SHetA2 and GSK525762, SHetA2 and INCB057643, SHetA2 and dBET6, SHetA2 and Alobresib, SHetA2 and Inobrodib, SHetA2 and BI-2536, SHetA2 and LB-100, SHetA2 and SMAP, SHetA2 and FTY720, SHetA2 and OP449, SHetA2 and perphenazine, SHetA2 and KPT-6566, SHetA2 and ATRA, SHetA2 and BJP-06-005-3, SHetA2 and Sulfopin, SHetA2 and AZD1208, SHetA2 and TP-3654, SHetA2 and PIM447, SHetA2 and SGI-1776, SHetA2 and LGH447, SHetA2 and SEL24, SHetA2 and Everolimus, SHetA2 and Paxalisib, SHetA2 and ATG-008, SHetA2 and Gedatolisib, SHetA2 and Samotolisib, SHetA2 and Sapanisertib, SHetA2 and Apitolisib, SHetA2 and Zotatifin, SHetA2 and TAK-981, SHetA2 and MRT-2359, and combinations thereof.
